(19) United States
(12) Patent Application Publication
(10) Pub. No.: US 2025/0144395 A1
(43) Pub. Date: May 8, 2025

(54) SYSTEMS AND METHODS FOR TRANSFEMORAL CAROTID INTERVENTION

(71) Applicant: Silk Road Medical, Inc., Sunnyvale, CA (US)

(72) Inventors: Bill Whealon, Sunnyvale, CA (US); Brad Steele, Sunnyvale, CA (US); Shanna Klein, Sunnyvale, CA (US); Abigail Brooks, Sunnyvale, CA (US); Zack Tegels, Sunnyvale, CA (US)

(21) Appl. No.: 18/933,413

(22) Filed: Oct. 31, 2024

Related U.S. Application Data
(60) Provisional application No. 63/569,390, filed on Mar. 25, 2024.
(60) Provisional application No. 63/595,988, filed on Nov. 3, 2023.

(51) Int. Cl. A61M 60/109; A61M 25/10; A61M 29/00; A61M 60/279; A61M 60/30

(52) U.S. Cl. CPC A61M 60/109; A61M 25/10; A61M 29/00; A61M 60/279; A61M 60/30; A61M 2025/1052; A61M 2202/0021; A61M 2202/0413; A61M 2205/106; A61M 2205/3334; A61M 2205/583; A61M 2206/10; A61M 2210/12

(57) ABSTRACT

A system is used to access and treat a carotid artery. The system includes an arterial access device that receives blood flow from the common carotid artery via a distal region of the arterial access device. A shunt fluidly connects to the arterial access device and defines a blood flow pathway for blood to flow from the arterial access device to a return site. A flow control assembly assists blood flow through the shunt and includes a pump having at least one roller that interacts with the tubing of the shunt to pump blood through the shunt toward the return site.

### CROSS REFERENCE TO RELATED APPLICATIONS

This application claims priority to U.S. patent application No. 63/595,988 filed Nov. 3, 2023, and U.S. patent application No. 63/569,390 filed Mar. 25, 2024, the contents of which are hereby incorporated by reference herein in their entireties.

### BACKGROUND

The present disclosure relates generally to medical methods and devices. More particularly, the present disclosure relates to methods, system, and devices for accessing and treating vasculature such as carotid arterial vasculature and optionally establishing retrograde blood flow during performance of carotid artery stenting and other procedures.

The present disclosure also relates to methods and systems for accessing and treating the cerebral arterial vasculature such as for the treatment of stroke, Intracranial Atherosclerotic Disease (ICAD), transient ischemic attack (TIA), acute ischemic stroke (AIS), tandem lesions, ruptured and unruptured intra- and extra-cranial aneurysm embolization, chronic occlusions, and other disease conditions of the neurovasculature.

### SUMMARY

The disclosed systems and methods establish and facilitate retrograde or reverse flow blood circulation such as for example in the region of the carotid artery bifurcation such as to limit or prevent the release of emboli into the cerebral vasculature, such as into the internal carotid artery.

In one aspect, there is disclosed a system for use in accessing and treating a carotid artery, the system comprising: an arterial access device adapted to be introduced into a common carotid artery via a percutaneous access location in a groin of a patient and receive blood flow from the common carotid artery via a distal region of the arterial access device, wherein the arterial access device includes a distal sheath which enters the common carotid artery; a shunt fluidly connected to the arterial access device, wherein the shunt comprises tubing defining a blood flow pathway for blood to flow from the arterial access device to a return site; and a flow control assembly coupled to the shunt and adapted to assist blood flow through the shunt, the flow control assembly including a pump having at least one roller that interacts with the tubing of the shunt to pump blood through the shunt toward the return site.

In another aspect, there is disclosed a method of treating a carotid artery, comprising: percutaneously accessing a femoral artery by inserting an arterial access device into the femoral artery through a puncture in the femoral artery, the arterial access device comprises a distal sheath; introducing the distal sheath into a common carotid artery via an aortic arch; expanding a balloon on the distal sheath to occlude at least a portion of the common carotid artery to cause blood to flow in a retrograde direction from an internal carotid artery into the distal sheath; shunting blood from the sheath to a return site via a shunt fluidly coupled to the distal sheath; and assisting blood flow through the shunt using a pump having at least one roller that interacts with a tubing of the shunt to pump blood through the shunt toward the return site.

Other features and advantages should be apparent from the following description of various embodiments, which illustrate, by way of example, the principles of the disclosure.

### BRIEF DESCRIPTION OF THE DRAWINGS

FIG. 1 is a schematic illustration of a retrograde blood flow system wherein an arterial access device accesses the common carotid artery via a transfemoral access location and a venous return device communicates with the femoral vein.

FIG. 2 is an enlarged view of the carotid artery wherein the carotid artery is occluded with an occlusion element on the sheath and connected to a reverse flow shunt, and an interventional device, such as a stent delivery system or other working catheter, is introduced into the carotid artery via an arterial access device.

FIG. 3 illustrates a cerebral circulation diagram including the Circle of Willis.

FIGS. 4A-6B illustrate arterial access devices useful in the methods and systems of the present disclosure.

FIGS. 7A and 7B illustrate an additional implementation of an arterial access device.

FIGS. 7C and 7D illustrate an implementation of a valve on the arterial access device.

FIGS. 8A through 8C and FIG. 9 illustrate implementations of a venous return device useful in the methods and systems of the present disclosure.

FIG. 10 schematically illustrates the system of FIG. 1 including a flow control assembly.

FIGS. 11-12 illustrate an implementation of a variable flow resistance component useful in the methods and systems of the present disclosure.

FIG. 13 shows an example flow controller housing.

FIG. 14 shows an example filter system.

FIGS. 15 and 16 show example sheath shapes.

FIG. 17 shows a schematic, cross-sectional view of at least a portion of a wall of a sheath.

FIG. 18 shows a schematic representation of a flow assistance system.

FIG. 19 shows a schematic representation of an example flow assist mechanism of an external flow circuit of the flow assistance system.

FIGS. 20 and 21 show example pumps.

FIGS. 22-24B show example roller pumps.

FIGS. 25-27 show an additional implementation of a roller pump.

FIGS. 28 and 29 show additional schematic representations of the flow assistance system.

FIGS. 30A-32 show an implementation of a vacuum flow resistor and components thereof.

FIGS. 33A and 33B show an implementation of a vacuum flow resistor that includes a vacuum breaker valve.

FIGS. 34 and 35 show an implementation of a high low switch system configuration which is configured to switch flow between a low flow state and a high flow state.

FIG. 36 shows a schematic representation of the flow assistance system wherein at least a portion of the external flow circuit is logic controlled.

FIGS. 37-38 show additional schematic representations of fluid pump systems.

FIG. 39 shows an implementation of an arterial access system.

FIG. 40 shows an example implementation of a dilator.

FIGS. 41 and 42 show enlarged views of distal tip regions of the dilator.

FIG. 43 shows an enlarged view of a proximal hub of the arterial access system.

FIG. 44 shows a portion of the proximal hub in cross section.

FIGS. 45 and 46 shows a distal region of a sheath in cross-section at a location where a distal end of a balloon attaches to the sheath.

FIGS. 47 and 48 shows cross-sectional views of the sheath in regions of a tack weld.

FIGS. 49 and 50 shows a region of the sheath in cross-section at a location where a proximal end of the balloon attaches to the sheath.

FIG. 51 shows a schematic representation of the sheath showing variations in flexibility.

FIGS. 52 and 53 shows a schematic representation of the sheath and dilator showing variations in flexibility.

### DETAILED DESCRIPTION

The disclosed systems and methods establish and facilitate retrograde or reverse flow blood circulation such as for example in the region of the carotid artery bifurcation such as to limit or prevent the release of emboli into the cerebral vasculature, such as into the internal carotid artery. In non-limiting examples, the methods are useful for interventional procedures, such as stenting and angioplasty, atherectomy, performed through a transfemoral access approach into the common carotid artery, either using an open surgical technique or using a percutaneous technique, such as a modified Seldinger technique or a micropuncture technique. Any of a wide variety of interventions can be performed in conjunction with the systems and method described herein including the treatment of stroke or other condition, Intracranial Atherosclerotic Disease (ICAD), transient ischemic attack (TIA), acute ischemic stroke (AIS), tandem lesions, ruptured and unruptured intra- and extra-cranial aneurysm embolization, chronic occlusions, Intravascular Lithotripsy (IVL), Shockwave Intravascular Lithotripsy (IVL), embolization, and other disease conditions of the neurovasculature or vasculature in general.

Access to the common carotid artery is established by placing an access sheath or other tubular access cannula into a lumen of an artery (such as the femoral artery) at an access location. The access location is primarily described herein as the femoral artery although other access locations are possible including the neck (such as via the common carotid artery), transradial access, transaxillary access (including radial and brachial access.) The artery can be accessed at the access location via a percutaneous access or via a surgical cut down.

A distal end or region of the sheath, catheter, or other elongated body is routed through the vasculature and positioned in communication with the common carotid artery such as proximal to the junction or bifurcation B from the common carotid artery to the internal and external carotid arteries. The distal region can be positioned within the common carotid artery or in communication with the common carotid artery. A percutaneous version of the sheath may have an occlusion member at the distal end, for example a compliant occlusion balloon or other component. A catheter or guidewire with an occlusion member, such as a balloon, may be placed through the access sheath and positioned in the proximal external carotid artery ECA to inhibit the entry of emboli, but occlusion of the external carotid artery is not necessary. A second return sheath is placed in the venous system, for example the internal jugular vein IJV or femoral vein FV. The arterial access and venous return sheaths are connected to create an external arterial-venous shunt. The balloon can include a radiopaque material for visualization. The balloon can also be at least partially covered by a sheath, collar or other element that can protect the balloon during percutaneous entry and that can be peeled away after use.

Retrograde flow at least in the carotid artery region is established and may be modulated. Flow through the common carotid artery is occluded, such as by using an internal occlusion member such as a balloon, or other type of occlusion means. When flow through the common carotid artery is blocked, the natural pressure gradient between the internal carotid artery and the venous system (or other location) causes blood to flow in a retrograde or reverse direction from the cerebral vasculature through the internal carotid artery and through the shunt into the venous system. In an alternate implementation, a vascular clamp can also be used via a surgical incision.

Alternately, the venous sheath can be eliminated and the arterial sheath is connected to an external collection reservoir or receptacle. The reverse flow can be collected in this receptacle. If desired, the collected blood can be filtered and subsequently returned to the patient during or at the end of the procedure. The pressure of the receptacle can be open to atmospheric pressure, causing the pressure gradient to create blood to flow in a reverse direction from the cerebral vasculature to the receptacle or the pressure of the receptacle could be a negative pressure.

Optionally, to achieve or enhance reverse flow from the internal carotid artery, flow from the external carotid artery may be blocked, such as by deploying a balloon or other occlusion element in the external carotid just above (i.e., distal) the bifurcation within the internal carotid artery.

Although the procedures and protocols are described herein in the context of carotid stenting, it will be appreciated that the methods for accessing the carotid artery described herein can also be useful for angioplasty, atherectomy, and any other interventional procedures which might be carried out in the carotid arterial system, such as at a location near the bifurcation between the internal and external carotid arteries. In addition, it will be appreciated that some of these access, vascular closure, and embolic protection methods will be applicable in other vascular interventional procedures, for example the treatment of acute stroke. The systems and methods described herein can also be used pursuant to a pulmonary procedure, a contrast removal procedure, peripheral atherectomy, transcatheter aortic valve replacement (TAVR), and thoracic endovascular aortic repair (TEVAR).

The present disclosure includes aspects for improving the performance of carotid artery access protocols. At least some of these aspects can be performed individually or in combination with one or more other of the improvements in order to facilitate and enhance the performance of the particular interventions in the carotid arterial system.

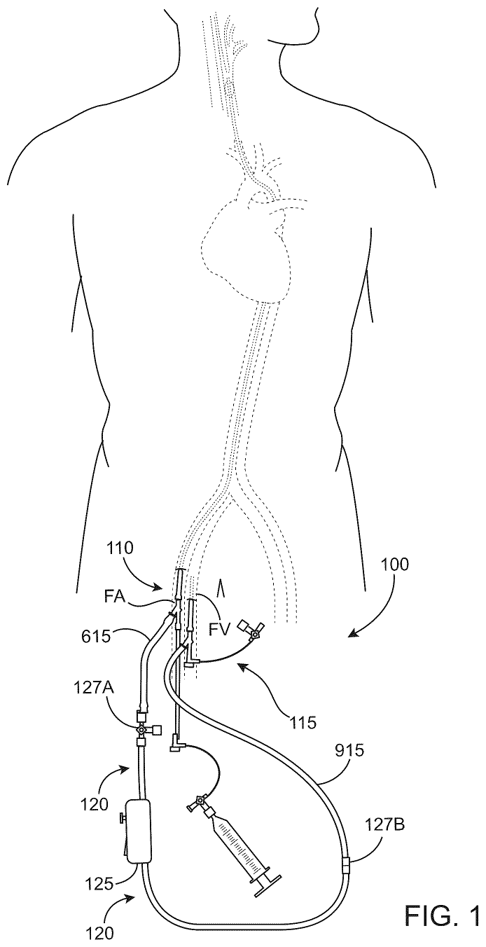

FIG. 1 shows a first implementation of a retrograde flow system 100 that is adapted to establish and facilitate retrograde or reverse flow blood circulation in the region of the carotid artery bifurcation to limit or prevent the release of emboli into the cerebral vasculature, particularly into the internal carotid artery. In an implementation, emboli removal occurs under radiofrequency (RF) guidance. An arterial access device 110 accesses the common carotid artery via a femoral approach via an access location at the femoral artery in the groin area. According to the femoral approach, the arterial access device 110 approaches the common carotid artery CCA via a percutaneous puncture into the femoral artery FA, such as in the groin, and up the aortic arch AA into the target common carotid artery CCA. The venous return device 115 can communicate with the jugular vein JV, the femoral vein FV or other vein. The venous return device 115 can be inserted into a central vein such as the femoral vein FV via a percutaneous puncture such as in the groin.

The system 100 interacts with the carotid artery to provide retrograde flow from the carotid artery to a venous return site, such as the internal jugular vein (or to another return site such as another large vein or an external receptacle in alternate implementations.) As described in more detail below, the retrograde flow system 100 includes an arterial access device 110, a venous return device 115, and a shunt 120 that provides a passageway for retrograde flow from the arterial access device 110 to the venous return device 115.

A flow control assembly 125 interacts with the shunt 120. The flow control assembly 125 is adapted to regulate and/or monitor the retrograde flow from the common carotid artery to the internal jugular vein, as described in more detail below. The flow control assembly 125 interacts with the flow pathway through the shunt 120, either external to the flow path, inside the flow path, or both. The arterial access device 110 provides direct or indirect access to the common carotid artery such as by at least partially inserting into the common carotid artery CCA via the access location. The arterial access device in an implementation has may a flared proximal end such as just adjacent to a hub of the arterial access device.

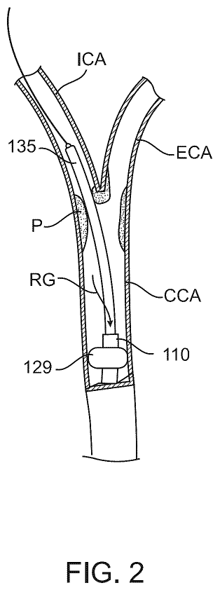

The venous return device 115 at least partially inserts into a venous return site such as the internal femoral vein FV. The arterial access device 110 and the venous return device 115 couple to the shunt 120 at connection locations 127a and 127b. When flow through the common carotid artery is blocked, the natural pressure gradient between the internal carotid artery and the venous system causes blood to flow in a retrograde or reverse direction RG (FIG. 2) from the cerebral vasculature through the internal carotid artery and through the shunt 120 into the venous system. The flow control assembly 125 modulates, augments, assists, monitors, and/or otherwise regulates the retrograde blood flow.

In an implementation, transfemoral access to the common carotid artery is achieved percutaneously via an incision or puncture in the skin in the region of the femoral artery through which the arterial access device 110 is inserted. If an incision is used, then the incision can be about 0.5 cm in length in a non-limiting example. An occlusion element, such as an expandable balloon, can be used to occlude the common carotid artery CCA at a location proximal of the distal end of the arterial access device 110. The occlusion element can be located on the arterial access device 110 or it can be located on a separate device.

In another implementation, the arterial access device 110 accesses the common carotid artery CCA via a percutaneous transcarotid approach with an access location in the neck region of a patient. Transcarotid access provides a short length and non-tortuous pathway from the vascular access point to the target treatment site. For a transcarotid approach, an arterial distance from the arteriotomy to the target treatment site (as measured traveling through the artery) is 15 cm or less. In an implementation, the distance is between 5 and 10 cm. In an alternate implementation, the arterial access device 110 accesses the common carotid artery CCA via a direct surgical transcarotid approach. In the surgical approach, the common carotid artery can be occluded using a tourniquet 2105.

With reference to the enlarged view of the carotid artery in FIG. 2, a therapeutic or interventional device, such as a stent delivery system 135 or other working catheter, can be introduced into the carotid artery via the arterial access device 110 or percutaneous sheath, as described in detail below. The stent delivery system 135 can be used to treat the plaque P such as to deploy a stent into the carotid artery. The arrow RG in FIG. 2 represents the direction of retrograde flow.

### DESCRIPTION OF EXAMPLE ANATOMY

### Collateral Brain Circulation

The Circle of Willis CW is the main arterial anastomatic trunk of the brain where all major arteries which supply the brain, namely the two internal carotid arteries (ICAs) and the vertebral basilar system, connect. The blood is carried from the Circle of Willis by the anterior, middle and posterior cerebral arteries to the brain. This communication between arteries makes collateral circulation through the brain possible. Blood flow through alternate routes is made possible thereby providing a safety mechanism in case of blockage to one or more vessels providing blood to the brain. The brain can continue receiving adequate blood supply in most instances even when there is a blockage somewhere in the arterial system (e.g., when the ICA is ligated as described herein). Flow through the Circle of Willis ensures adequate cerebral blood flow by numerous pathways that redistribute blood to the deprived side.

The collateral potential of the Circle of Willis is believed to be dependent on the presence and size of its component vessels. It should be appreciated that considerable anatomic variation between individuals can exist in these vessels and that many of the involved vessels may be diseased. For example, some people lack one of the communicating arteries. If a blockage develops in such people, collateral circulation is compromised resulting in an ischemic event and potentially brain damage. In addition, an autoregulatory response to decreased perfusion pressure can include enlargement of the collateral arteries, such as the communicating arteries, in the Circle of Willis. An adjustment time is occasionally required for this compensation mechanism before collateral circulation can reach a level that supports normal function. This autoregulatory response can occur over the space of 15 to 30 seconds and can only compensate within a certain range of pressure and flow drop. Thus, it is possible for a transient ischemic attack to occur during the adjustment period. Very high retrograde flow rate for an extended period of time can lead to conditions where the patient's brain is not getting enough blood flow, leading to patient intolerance as exhibited by neurologic symptoms or in some cases a transient ischemic attack.

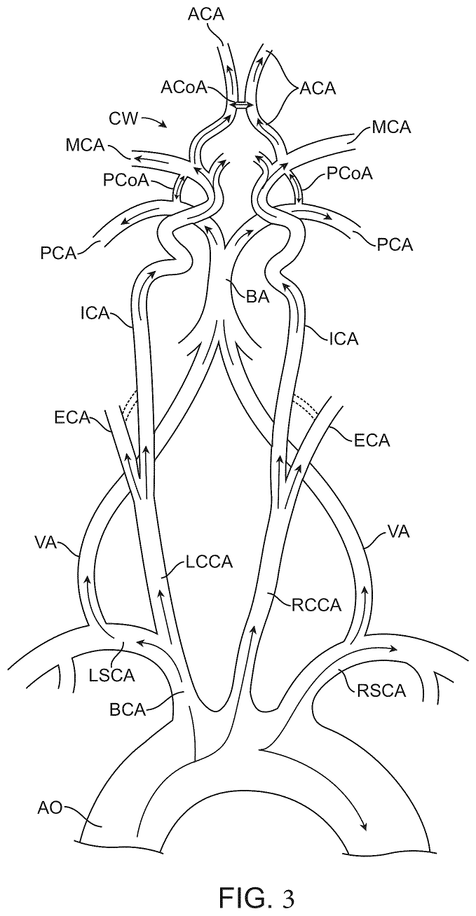

FIG. 3 depicts a normal cerebral circulation and formation of Circle of Willis CW. The aorta AO gives rise to the brachiocephalic artery BCA, which branches into the left common carotid artery LCCA and left subclavian artery LSCA. The aorta AO further gives rise to the right common carotid artery RCCA and right subclavian artery RSCA. The left and right common carotid arteries CCA gives rise to internal carotid arteries ICA which branch into the middle cerebral arteries MCA, posterior communicating artery PcoA, and anterior cerebral artery ACA. The anterior cerebral arteries ACA deliver blood to some parts of the frontal lobe and the corpus striatum. The middle cerebral arteries MCA are large arteries that have tree-like branches that bring blood to the entire lateral aspect of each hemisphere of the brain. The left and right posterior cerebral arteries PCA arise from the basilar artery BA and deliver blood to the posterior portion of the brain (the occipital lobe).

Anteriorly, the Circle of Willis is formed by the anterior cerebral arteries ACA and the anterior communicating artery ACoA which connects the two ACAs. The two posterior communicating arteries PCoA connect the Circle of Willis to the two posterior cerebral arteries PCA, which branch from the basilar artery BA and complete the Circle posteriorly.

The common carotid artery CCA also gives rise to external carotid artery ECA, which branches extensively to supply most of the structures of the head except the brain and the contents of the orbit. The ECA also helps supply structures in the neck and face. The common carotid arteries are encased on each side in a layer of fascia called the carotid sheath. This sheath also envelops the internal jugular vein and the vagus nerve. Anterior to the sheath is the sternocleidomastoideole. Transcarotid access to the common carotid artery and internal jugular vein, either percutaneous or surgical, can be made immediately superior to the clavicle, between the two heads of the sternocleidomastoid muscle and through the carotid sheath, with care taken to avoid the vagus nerve.

The common carotid artery bifurcates into the internal and external carotid arteries. The internal carotid artery continues upward without branching until it enters the skull to supply blood to the retina and brain. The external carotid artery branches to supply blood to the scalp, facial, ocular, and other superficial structures. Intertwined both anterior and posterior to the arteries are several facial and cranial nerves. Additional neck muscles may also overlay the bifurcation. These nerve and muscle structures can be dissected and pushed aside to access the carotid bifurcation during a carotid endarterectomy procedure. In some cases the carotid bifurcation is closer to the level of the mandible, where access is more challenging and with less room available to separate it from the various nerves which should be spared.

### Retrograde Blood Flow System

As discussed, the retrograde flow system 100 includes the arterial access device 110, venous return device 115, and shunt 120 which provides a passageway for retrograde flow from the arterial access device 110 to the venous return device 115. The system also includes the flow control assembly 125, which interacts with the shunt 120 to regulate and/or monitor retrograde blood flow through the shunt 120. Example implementations of the components of the retrograde flow system 100 are now described.

### Arterial Access Device

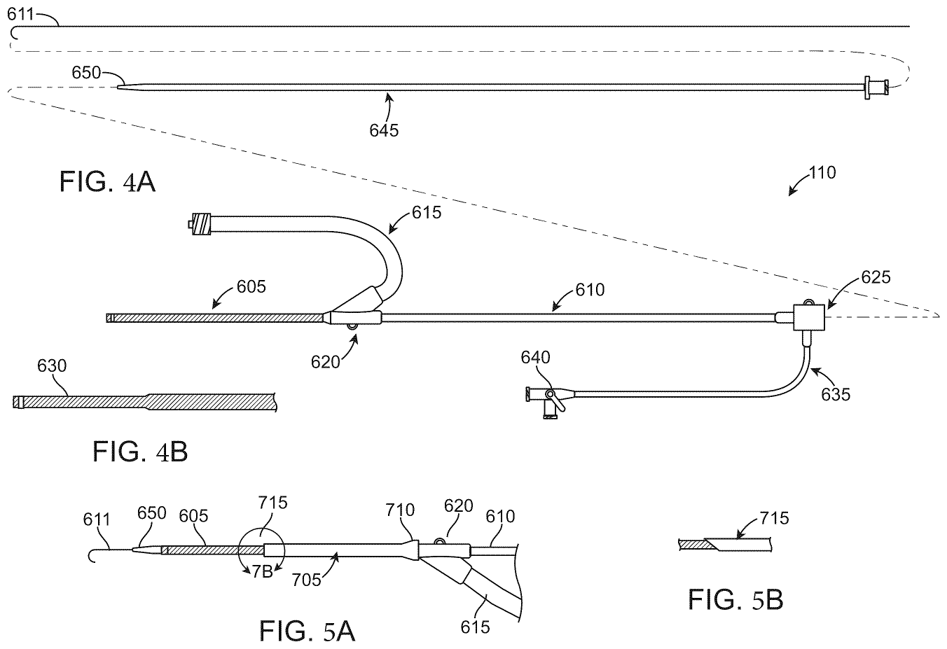

FIG. 4A shows an exemplary implementation of the arterial access device 110, which comprises a distal catheter or sheath 605, a proximal extension 610, a flow line 615, an adaptor or Y-connector 620, and a hemostasis valve 625. The arterial access device may also comprise a dilator 645 with a tapered tip 650 and an introducer guide wire 611. The arterial access device together with the dilator and introducer guidewire are used together to gain access to a vessel. Features of the arterial access device may be optimized for transfemoral access. The arterial access device or any portion thereof can also be referred to as a catheter.

The distal sheath 605 is adapted to be introduced through a percutaneous incision or puncture in a wall of an artery (such as the femoral artery in the area of the groin). The distal sheath is positioned up the aortic arch AA into the target common carotid artery CCA. The sheath can vary in size and can be for example an 8 French sheath.

The distal sheath 605 can have a stepped or other configuration having a reduced diameter distal region 630 that forms one or more steps or gradual transitions along the length of the distal sheath, as shown in FIG. 4B, which shows an enlarged view of the distal region 630 of the sheath 605. The change in diameter can occur in the inner diameter, the outer diameter, or both the inner diameter and outer diameter of the distal sheath. During use, the distal sheath 605 can be positioned in a blood vessel such that the step aligns with a predetermined location of a blood vessel. The reduced diameter section 630 also permits a reduction in size of the arteriotomy for introducing the sheath 605 into the artery while having a minimal impact in the level of flow resistance. Further, the reduced distal diameter section may be more flexible and thus more conformal to the lumen of the vessel.

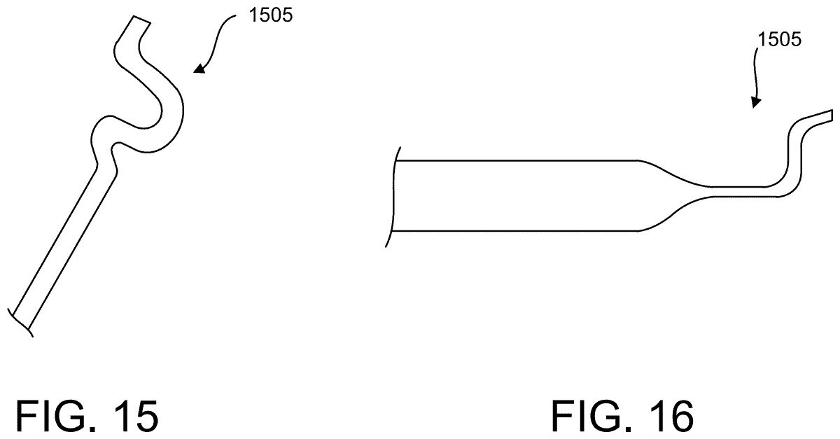

At least a portion of the arterial access device and/or the dilator 645 can be pre-shaped in any of a variety of shapes to facilitate navigation through the blood vessel. For example, FIG. 15 shows a distal region 1505 of a sheath and/or dilator that has a pre-shaped region 1505. The region 1505 can have an undulating shape that forms a corresponding internal, undulating device pathway such as via an internal lumen (e.g., a guidewire lumen) in the sheath and/or dilator. In another implementation shown in FIG. 16, a distal region of the sheath and/or dilator is configured as a reduced diameter region 1505. That is, a diameter of the region 1505 reduces moving in a distal direction.

The arterial access device 110 can be formed of a layered outer wall wherein each layer has a predetermined flexibility/stiffness. In an implementation shown in FIG. 17, a wall of the arterial access device has an inner layer 6125, a middle layer 1630, and an outer layer 1635 (although the number of layers can vary.) In FIG. 17, different stiffnesses are represented by discrete regions of each layer. Each discrete region is schematically represented as a rectangle although the size and shape as well as relative size and shape of each region can vary. The stiffness of each layer can vary moving along the length of the arterial access device with the transitions in stiffness being offset between layers.

The arterial access device can include a blood flow, blood pressure, or blood flow velocity measuring component to measure and provide an indication to a user of such features. In a method of use, patient blood pressure is increased during a procedure to increase blood flow rate through the shunt. The system can also be configured to shut off or slow down reverse flow (such as by shutting off or slowing down a pump) if blood pressure falls below a threshold value.

The arterial access device 110 can include one or more portions that include colors or other visual indications to provide a user with an indication of a purpose or functionality of a portion of the arterial access device 110, such as a port. For example, a particular color can indicate that a port connects to a main lumen, an aspiration lumen, or a shunt lumen of the arterial access device. Or another color can coincide with a pump access port for example. Other colors on the arterial access device can indicate a specification of the distal sheath 605, such an inner diameter, outer diameter, stiffness and/or flexibility 125.

With reference again to FIG. 4A, the proximal extension 610, which is an elongated body, has an inner lumen which is contiguous with an inner lumen of the sheath 605. The lumens can be joined by the Y-connector 620 which also connects a lumen of the flow line 615 to the sheath. In the assembled system, the flow line 615 connects to and forms a leg of the retrograde shunt 120 (FIG. 1). The proximal extension 610 can have a length sufficient to space the hemostasis valve 625 well away from the Y-connector 620, which is adjacent to the insertion site.

A flush line 635 can be connected to the side of the hemostasis valve 625 and can have a stopcock 640 at its proximal or remote end. The flush-line 635 allows for the introduction of saline, contrast fluid, or the like, during the procedures. The flush line 635 can also allow pressure monitoring during the procedure. An elongated body such as a dilator 645 or other elongated body having a tapered distal end 650 can be provided to facilitate introduction of the distal sheath 605 into the artery or passage of the distal sheath 605 through the blood vessels. The dilator 645 can be introduced through the hemostasis valve 625 so that the tapered distal end 650 extends through the distal end of the sheath 605, as best seen in FIG. 5A. The dilator 645 can have a central lumen to accommodate a guide wire. Typically, the guide wire is placed first into the vessel, and the dilator/sheath combination travels over the guide wire as it is being introduced into the vessel. The dilator 645 can include one or more markings, symbols or other indicator that can provide a user with a visual indication of an insertion depth of the dilator into an artery. For example, the markings can be a graduated scale of markings.

The arterial access device can include or be coupled to a depth indicator that provide a visual indication of a depth of the device when inserted into an artery. The depth indicator, such as a collar on the arterial access device, can lock onto a portion of the arterial access device.

Optionally, a sheath stopper 705 such as in the form of a tube may be provided which is coaxially received over the exterior of the distal sheath 605, also as seen in FIG. 5A. The sheath stopper 705 is configured to prevent the sheath from being inserted too far into the vessel. The sheath stopper 705 is sized and shaped to be positioned over the sheath body 605 such that it covers a portion of the sheath body 605 and leaves a distal portion of the sheath body 605 exposed. The sheath stopper 705 may have a flared proximal end 710 that engages the adapter 620, and a distal end 715. Optionally, the distal end 715 may be beveled, as shown in FIG. 5B. The length of the sheath stopper 705 limits the introduction of the sheath 605 to the exposed distal portion of the sheath 605, as seen in FIG. 5A, such that the sheath insertion length is limited to the exposed distal portion of the sheath. The sheath stopper 705 may be removable from the sheath so that if the user desired a greater length of sheath insertion, the user could remove the sheath stopper 705, cut the length (of the sheath stopper) shorter, and re-assemble the sheath stopper 705 onto the sheath such that a greater length of insertable sheath length protrudes from the sheath stopper 705.

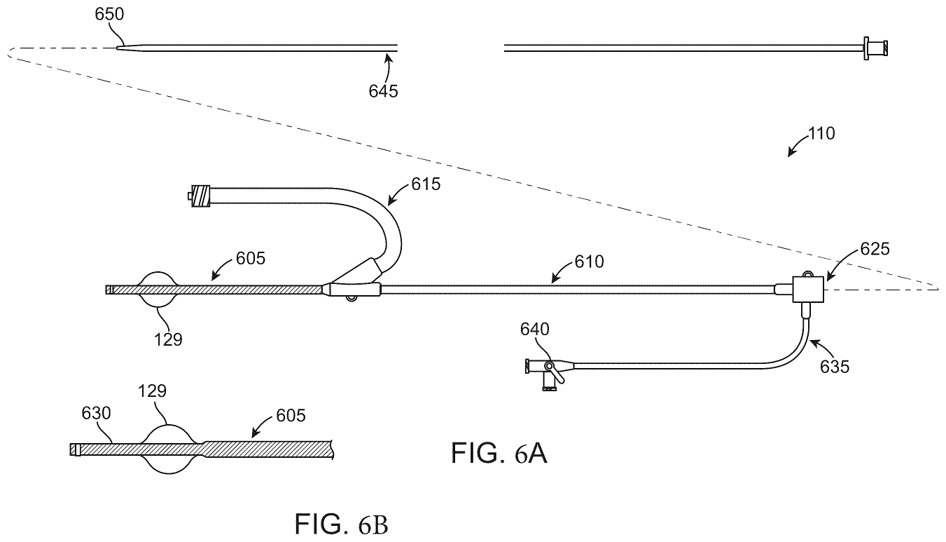

FIG. 6A shows another implementation of the arterial access device 110 wherein the distal sheath 605 includes an occlusion element 129 for occluding flow through an artery such as the common carotid artery. If the occluding element 129 is an inflatable structure such as a balloon or the like, the sheath 605 can include an inflation lumen that communicates with the occlusion element 129. The inflation lumen can be sized and shaped to removably or fixedly contain one or more wires or other elongated elements configured to shape the arterial access device 110 or configured to assist in steering the arterial access device through one or more blood vessels. The occlusion element 129 can be an inflatable balloon, but it could also be an inflatable cuff, a conical or other circumferential element which flares outwardly to engage the interior wall of the common carotid artery to block flow there past, a membrane-covered braid, a slotted tube that radially enlarges when axially compressed, or similar structure which can be deployed by mechanical means, or the like. In the case of balloon occlusion, the balloon can be compliant, non-compliant, elastomeric, reinforced, or have a variety of other characteristics.

In an implementation, the balloon is an elastomeric balloon which is closely received over the exterior of the distal end of the sheath prior to inflation. When inflated, the elastomeric balloon can expand and conform to the inner wall of the common carotid artery (or other artery where it is positioned). In an implementation, the elastomeric balloon is able to expand to a diameter at least twice that of the non-deployed configuration, frequently being able to be deployed to a diameter at least three times that of the undeployed configuration, more preferably being at least four times that of the undeployed configuration, or larger.

As shown in FIG. 6B, the distal sheath 605 with the occlusion element 129 can have a stepped or other configuration having a reduced diameter distal region 630. The distal region 630 can be sized for insertion into the carotid artery with the remaining proximal region of the sheath 605 having larger outside and luminal diameters.

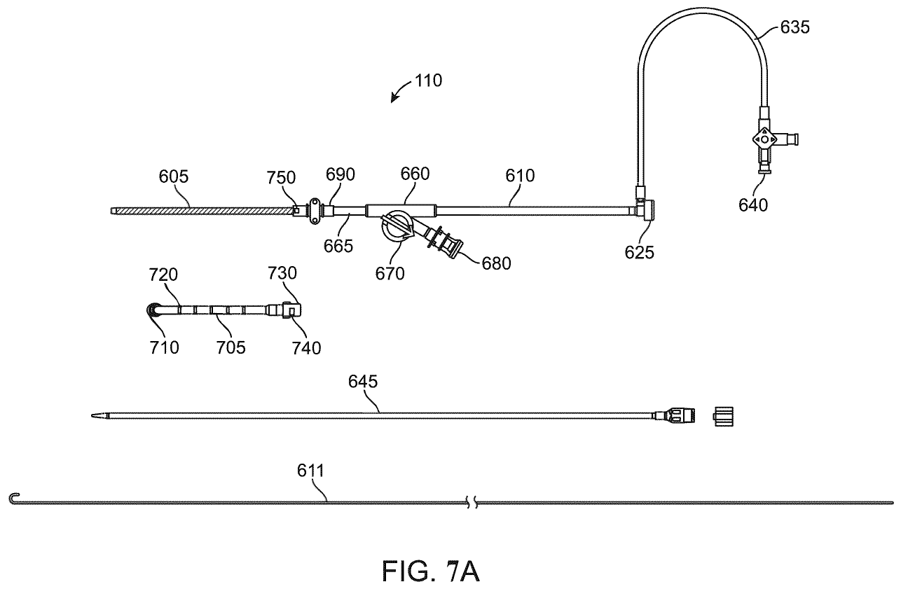

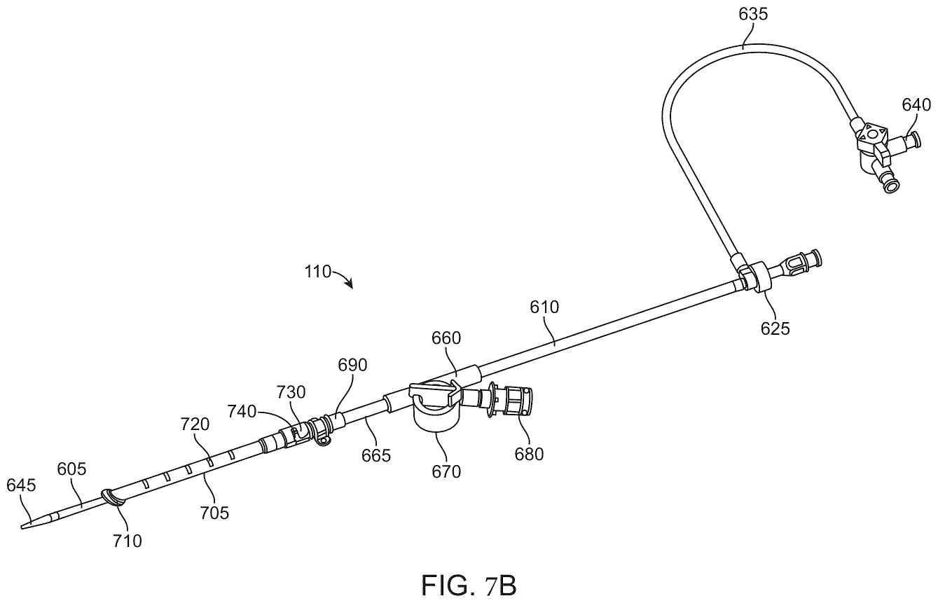

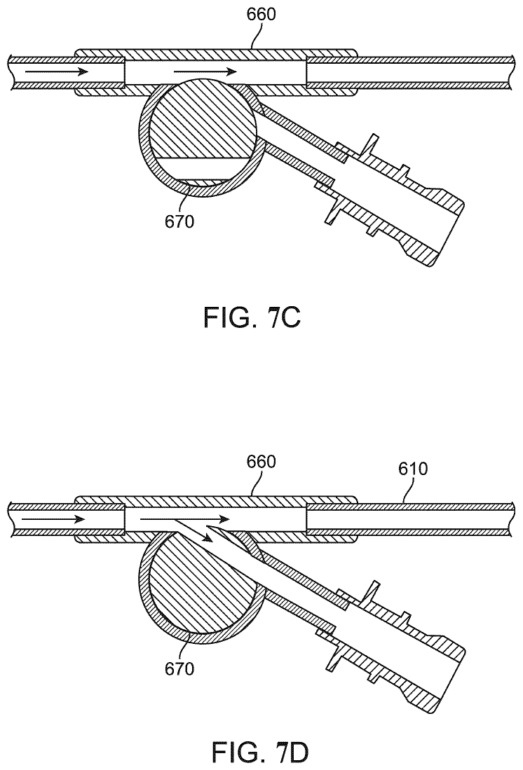

Another arterial access device is shown in FIGS. 7A-7D. FIG. 7A shows the components of the arterial access device 110 including arterial access sheath 605, sheath dilator 645, sheath stopper 705, and sheath guidewire 611. FIG. 7B shows the arterial access device 110 as it would be assembled for insertion over the sheath guide wire 611 into the carotid artery. After the sheath is inserted into the artery and during the procedure, the sheath guide wire 611 and sheath dilator 705 are removed. In this configuration, the sheath has a sheath body 605, proximal extension 610, and proximal hemostasis valve 625 with flush line 635 and stopcock 640. The proximal extension 610 extends from a Y-adapter 660 to the hemostasis valve 625 where the flush line 635 is connected. The sheath body 605 is the portion that is sized to be inserted into the artery and is actually inserted into the artery during use.

The sheath has a Y-adaptor 660 that connects the distal portion of the sheath to the proximal extension 610. The Y-adapter can also include a valve 670 that can be operated to open and close fluid connection to a connector or hub 680 that can be removably connected to a flow line such as a shunt. The valve 670 is positioned immediately adjacent to an internal lumen of the adapter 660, which communicates with the internal lumen of the sheath body 605. FIGS. 7C and 7D show non-limiting details in cross section of the Y-adaptor 660 with the valve 670 and the hub 680. FIG. 7C shows the valve closed to the connector. This is the position that the valve would be in during prep of the arterial sheath. The valve is configured so that there is no potential for trapped air during prep of the sheath. FIG. 7D shows the valve open to the connector. This position would be used once the flow shunt 120 is connected to hub 680, and would allow blood flow from the arterial sheath into the shunt. This configuration eliminates the need to prep both a flush line and flow line, instead allowing prep from the single flush line 635 and stopcock 640. This single-point prep is identical to prep of conventional introducer sheaths which do not have connections to shunt lines, and is therefore more familiar and convenient to the user. In addition, the lack of flow line on the sheath makes handling of the arterial sheath easier during prep and insertion into the artery.

The stopcock 640 can vary in size. In an implementation, the stopcock 640 has an internal lumen of about 0.200 inch although the size can vary. The stopcock or the sheath can be configured to achieve laminar blood flow therethrough. A stopcock as such can be positioned at one or more locations along the shunt such as for example between the arterial access device and the flow controller, and/or between the flow controller and venous return device.

With reference again to FIG. 7B, the sheath may also contain a second more distal connector 690, which is separated from the Y-adaptor 660 by a segment of tubing 665. In an implementation, the distal connector 690 contains suture eyelets to aid in securement of the sheath to the patient once positioned.

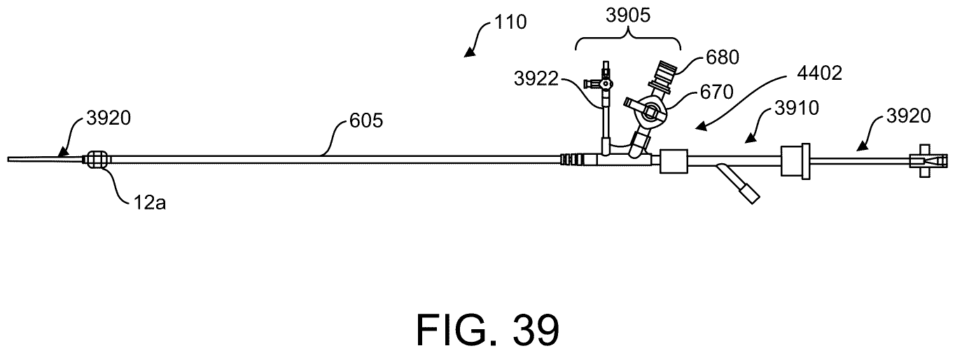

FIG. 39 shows an implementation of an arterial access system which includes an arterial access device 110 formed of a distal catheter or sheath 605 having a proximal hub 3905. A proximal access portion such as a rotatable hemostasis valve (RHV) 3910 is removably or fixedly attached to the proximal hub 3905. A dilator 3920 is sized and shaped to be inserted into the arterial access device 110 via insertion into the RHV 3910 such that the dilator 3920 extends into an internal lumen of the sheath 605 and such that a distal region of the dilator 3920 extends out of a distal end of the sheath 605 while a proximal region of the dilator 3920 extends proximally out of the RHV 3910. An expandable occlusion element 129, such as a balloon, for occluding flow through an artery such as the common carotid artery is located on a distal region of the sheath 605. If the occluding element 129 is an inflatable structure such as a balloon or the like, the sheath 605 can include an inflation lumen that communicates with the occlusion element 129 and that is accessed via an inflation port 3922 on the hub 3905.

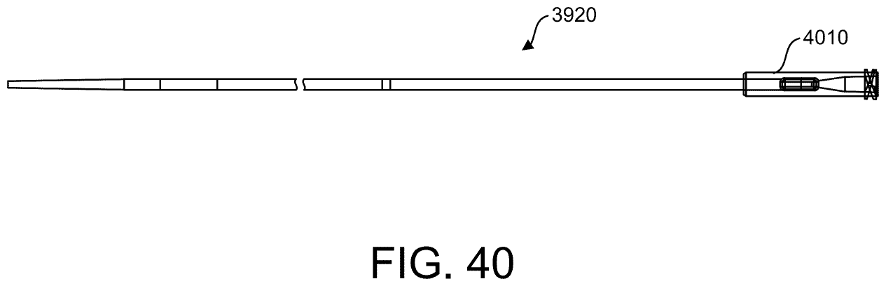

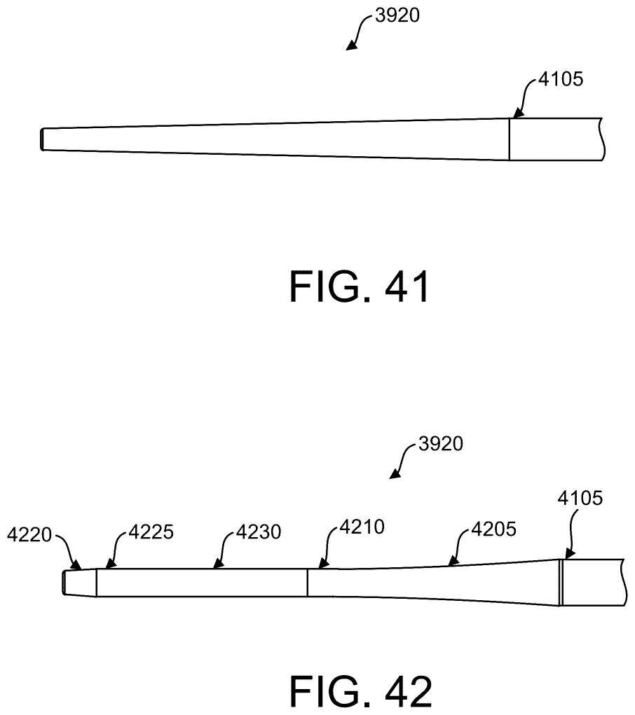

FIG. 40 shows an example implementation of the dilator 3920, which is an elongated body sized and shaped to fit within the internal lumen of the arterial access device 110. The dilator 3920 has an internal lumen sized and shaped to receive a guidewire. FIG. 41 shows an enlarged view of a distal tip region of the dilator 3920. The distal region tapers from a taper location 4105 to a distal tip of the dilator 3920. The taper can comprise a uniform reduction in diameter moving from the location 4105 to the distal tip. In an implementation, the taper region has a length of 1-6 cm. FIG. 42 shows an enlarged view of another implementation of the distal tip region of the dilator 3920. This implementation includes two regions of taper including a first taper region 4205 that starts to taper at location 4105 and tapers to a location 4210. A second taper region 4220 starts to taper at a location 4225 and tapers to a distal edge of the dilator 3920. A region 4230 of uniform outer diameter is located between the first taper region 4205 and the second taper region 4220. The dilator 3920 can include additional taper regions. The double taper configuration of FIG. 42 provides increased flexibility for the distal region compared to the implementation of FIG. 41.

During use, a user can visually track the location of the sheath 110 and the dilator 3920 as they are positioned into the artery. The sheath 110 and the dilator 3920 are both radiopaque such that they can be visualized via fluoroscopy. A first optical marker is located on a proximal end region of the dilator 3920. When the first optical marker is aligned with a landmark of the sheath 110, such as the proximal end of the RHV 3910, such alignment between the optical marker and the landmark on the sheath is an indication that the dilator's distalmost tapered region is fully extending from a distal tip of the sheath 110 such that the beginning of the taper is located at the distal tip of the sheath. That is, the alignment indicates to a user that the location 4105 (FIGS. 41 and 42) where the dilator begins to taper is aligned with a distal most end of the sheath 110.

This can be an important alignment position such that there is no gap present between the outer diameter of the dilator and the inner diameter of the sheath. If the dilator is shifted proximally or distally from this position, a gap will be present between the sheath and dilator which may cause potential for vessel damage or disease embolization during tracking.

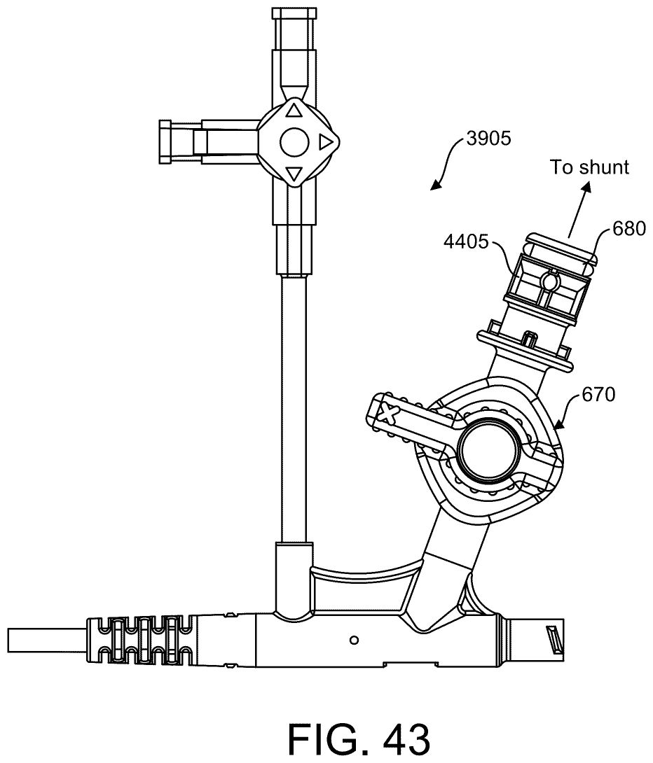

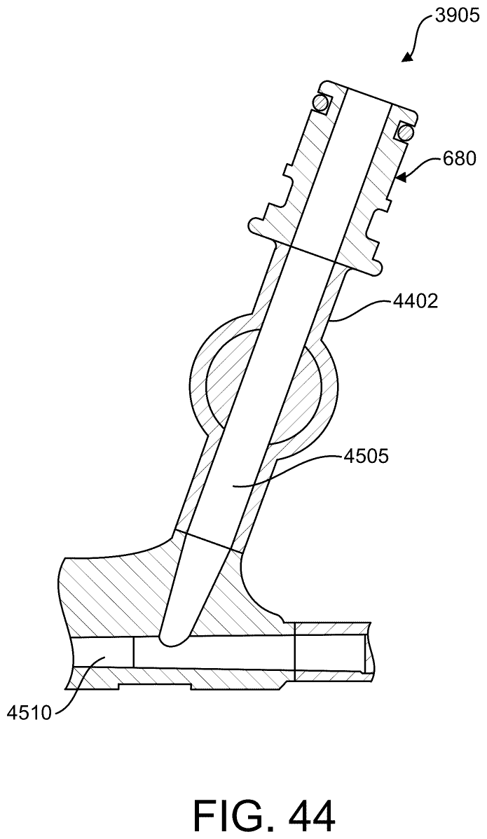

With reference again to FIG. 39, the proximal hub 3905 has a Y-adaptor that forms a shunt flow arm 4402 that communicates with the internal lumen of the sheath 605. The flow shunt arm 4402 has a valve 670 that can be operated to selectively open and close fluid connection to a connector 680 that can be removably connected to the shunt 120. FIG. 43 shows an enlarged view of the proximal hub 3905. FIG. 44 shows a portion of the hub 3905 in cross section. The shunt flow arm 4402 has an internal lumen 4505 that communicates with an internal lumen 4510 of the sheath 605. An internal diameter of the internal lumen 4505 within the hub 3905 increases in size from a diameter at the sheath inner lumen up to a diameter at the connector 680. The internal diameters of the internal lumens can vary. In a non-limiting example implementation, an internal diameter of the internal lumen 4505 is 0.113″ diameter where it connects to the sheath and it increases to 0.210″ at the connector 680.

During a carotid artery revascularization procedure, the arterial sheath 605 can be inserted into the common carotid artery (CCA) of the patient such as via a transfemoral approach wherein an access location for the sheath is located at the transfemoral artery. As described elsewhere herein, to achieve reverse flow of blood, the CCA may be occluded (such as using an expandable balloon as described below) to stop antegrade blood flow from the aorta through the CCA. Flow through the CCA can be occluded with an internal occlusion member such as a balloon, or other type of occlusion means. When flow through CCA is blocked, the natural pressure gradient between the internal carotid artery (ICA) and the venous system will cause blood to flow in a retrograde or reverse direction from the cerebral vasculature. Blood from the ICA and the external carotid artery (ECA) flows in a retrograde direction and the systems described herein allow the retrograde blood to flow into the sheath 605, through the flow controller 1130, the venous sheath 910, and then returned into the patient's femoral vein as described elsewhere herein. Loose embolic material can be carried with the retrograde blood flow into the arterial sheath 605.

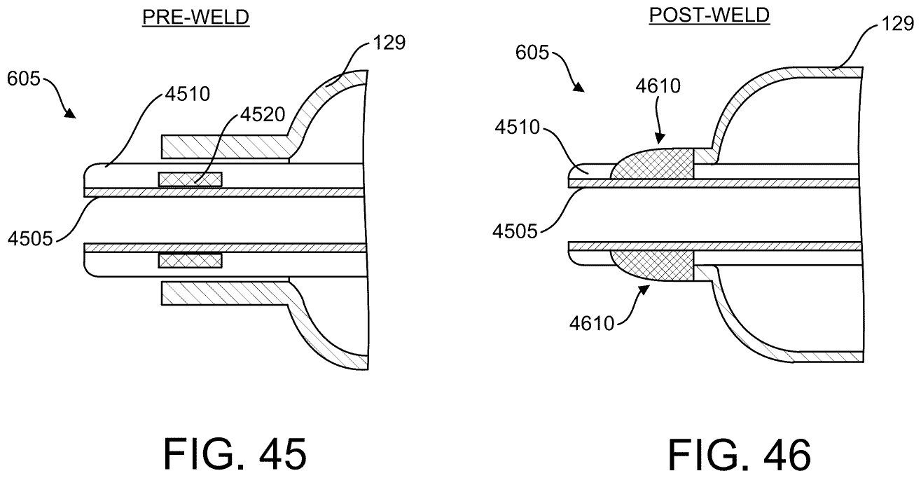

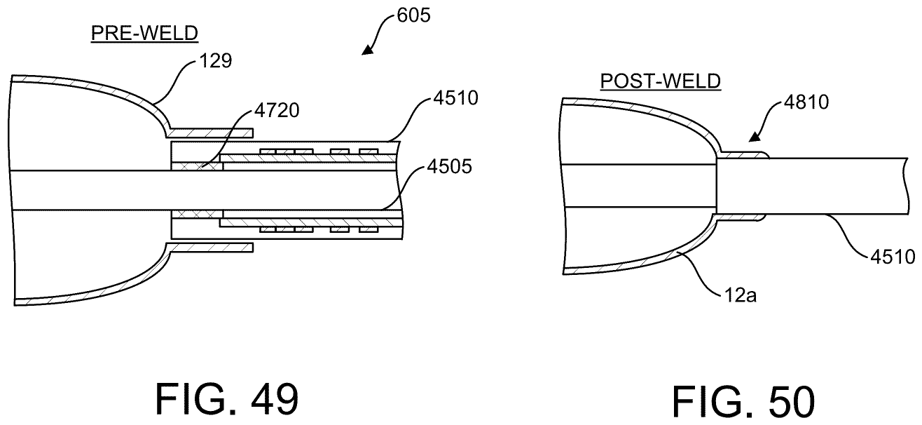

As discussed, an expandable occlusion element 129, such as a balloon 129, is located on a distal region of the sheath 605. FIG. 45 shows a distal region of the sheath 605 in cross-section at a location where a distal end of the balloon 129 attaches to the sheath 605. FIG. 45 shows the system in a pre-assembled or pre-formed configuration where the sheath 605 has not yet been fully assembled such that a weld has not been conducted to bond the components of the sheath and the balloon together. The sheath 605 is formed of an annular inner layer (such as a coil element) 4505 co-axially contained within an outer jacket 4510. A distal weld region 4520 attaches the inner layer 4505 to the outer jacket 4510 such that the inner layer 4505 is attached to the outer jacket 4510 via the weld region 4520 at a location distal of the balloon 129. A second, proximal tack weld region 4720 (FIG. 49) connecting the inner layer 4505 and the outer jacket 4510 is located at another region of the sheath 605 proximal to the balloon 129. The outer jacket and inner layer of the sheath are thus attached to one another at least at a distal weld location and at a proximal tack weld location. The positions of the distal weld location and at a proximal tack weld location along the length of the sheath 605 can vary. The distal weld region 4520 is a “full weld” with no interruptions, completely bonded such that fluid does not leak.

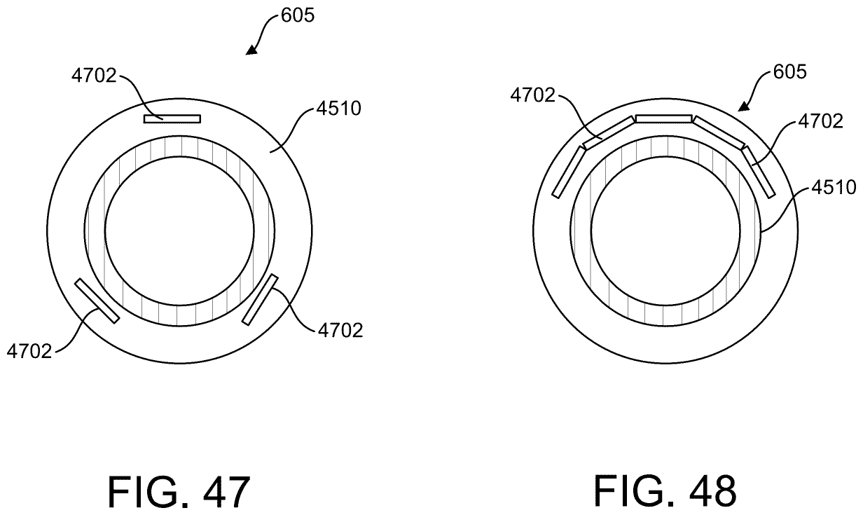

In the region of the sheath 605 between the distal weld location and the proximal tack weld location, the outer jacket and inner layer of the sheath are not fixedly attached such that an annular balloon inflation lumen is formed therebetween for inflation of the balloon 129. Such an inflation lumen is interrupted by the tack weld such that one or more lumens are needed through the tack weld for inflation of the lumen. FIG. 47 shows a cross-sectional view of the sheath 605 in the region of a tack weld. One or more inflation lumens 4702 are located in the tack weld region such as being interspersed around a circumference of the outer jacket 4510. In another implementation shown in FIG. 48, the inflation lumens 4702 are positioned along one side of the outer jacket 4510. Other configurations of inflation lumens are within the scope of this disclosure.

With reference again to FIG. 45, a distal end of the balloon 129 is positioned adjacent the outer jacket 4510. FIG. 46 shows the region after a weld has been performed such that a weld region 4610 attaches the balloon 129 to the inner layer 4505 of the sheath 605.

FIG. 49 shows a region of the sheath 605 in cross-section at a location where a proximal end of the balloon 129 attaches to the sheath 605. The tack weld region 4720 attaches the inner layer 4505 to the outer jacket 4510. FIG. 50 shows the region after a weld has been performed such that a weld region 4810 attaches the balloon 129 to the outer jacket 4510 of the sheath 605. The balloon is thus attached at a distal end to the inner layer 4505 (as shown in FIG. 46) while being attached at a proximal end to the outer jacket 4510 (as shown in FIG. 50.)

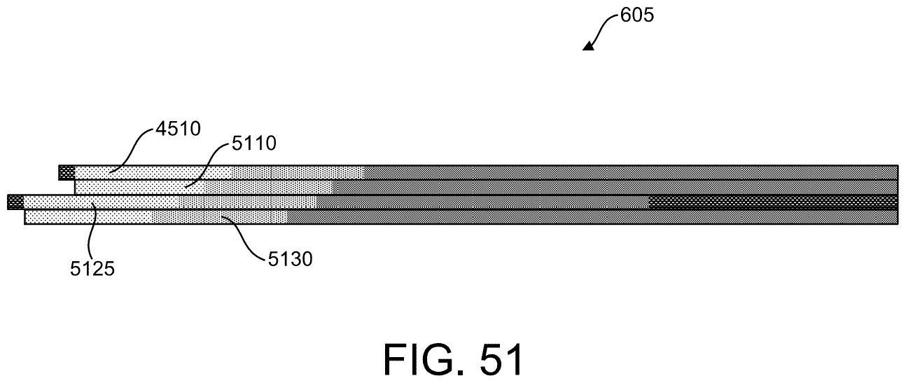

FIG. 51 shows a schematic representation of a wall of the sheath 605 extending from a rightmost proximal end to a leftmost distal end. The sheath 605 is formed of an outer layer comprising an outer jacket 4510 that surrounds a coil layer 5110. An inner layer is contained within the outer layer wherein the inner layer is formed of an inner jacket 5125 that surrounds a coil layer 5130. One or more material properties of any component of the outer layer and the inner layer can vary moving along a length of the sheath 605. For example, durometer and flexibility of one or more of the layers can vary moving along a length of the sheath 605. In FIG. 51, a durometer or stiffness of each layer is represented by a corresponding shade with darker shades representing higher durometer/stiffness (and lower flexibility) and lighter shades representing lower durometer/stiffness (and higher flexibility.) Each of the outer jacket 4510, coil layer 5110, inner jacket 5125 and coil layer 5130 has zones that increase in flexibility moving in a distal direction. However, the variations in flexibility are divided into zones that are offset from one another moving along the length of the sheath. Thus, one of the layers can have a flexibility that is different from a flexibility of another layer at any point along the length of the sheath 605.

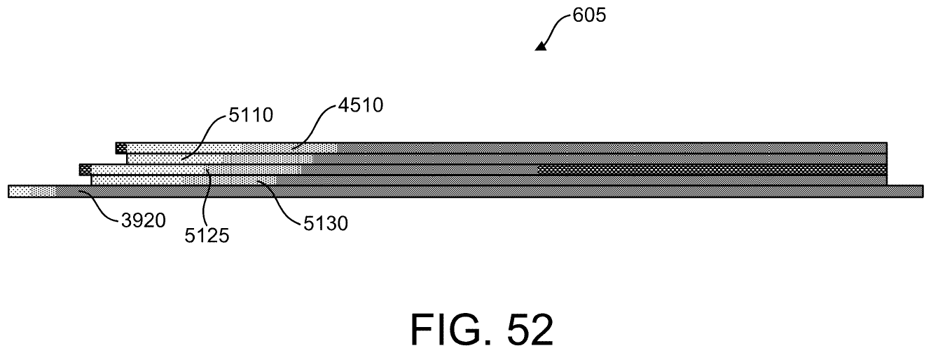

In an implementation, the dilator 3920 has a consistent durometer along a portion of its length such as along a majority of its length with a variation in flexibility in a distal region of the dilator. This is represented in FIG. 52, which shows a schematic representation of the dilator 3920 nested in the sheath 605. The region of the dilator 3920 that varies in flexibility does not overlap the regions of the sheath that vary in flexibility. Thus, the dilator 3920 can have a consistent flexibility along a portion of the length of the dilator 3920 that is nested inside the sheath 605 with a distal region of the dilator 3920 outside of the sheath 605 varying in flexibility.

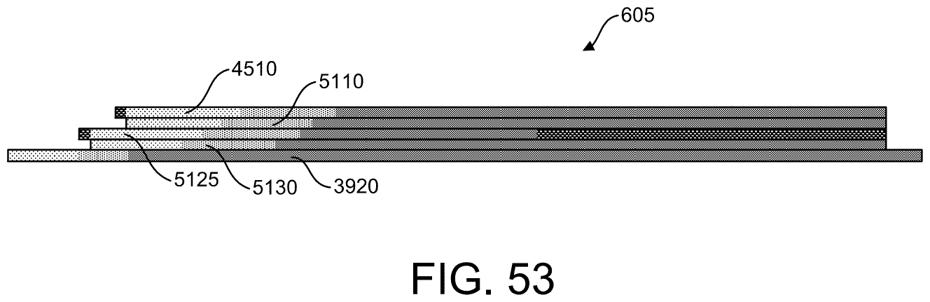

In another implementation represented in FIG. 53, the durometer variations at the distal end of the dilator are longer in length to allow more flexibility of the dilator to interact with the flexibility of the sheath. During use, the two components (dilator and sheath) can be moved relative to each other if needed to alter the flexibility of the system as a whole and the amount of support provided to the sheath by the dilator. The variation in flexibility of the distal end of the dilator may overlap the variation in sheath flexibility in this implementation.

### Venous Return Device

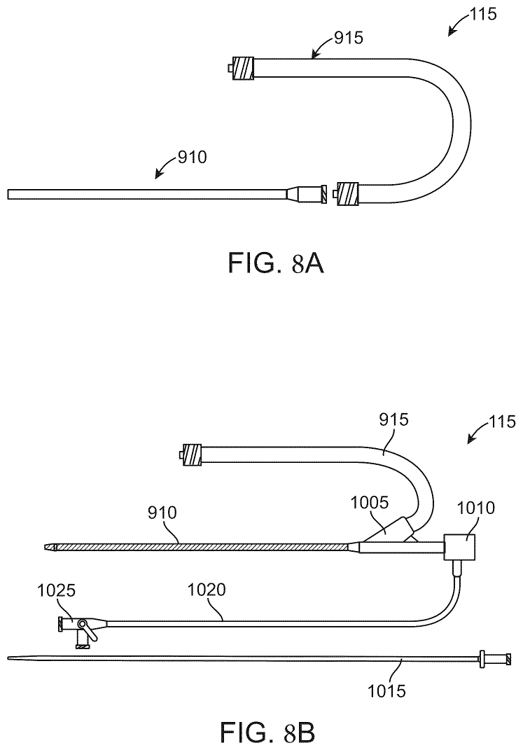

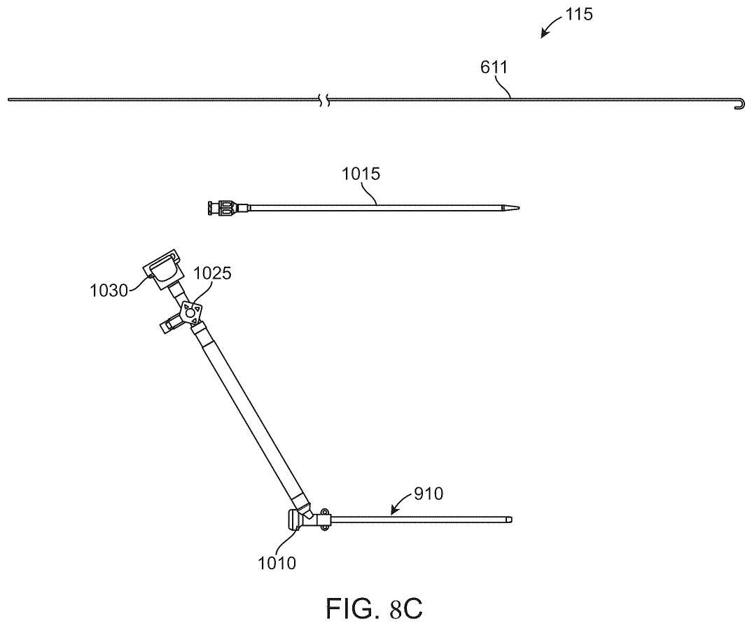

Referring now to FIGS. 8A and 8B, the venous return device 115 can comprise a distal sheath 910 and a flow line 915, which connects to and forms a leg of the shunt 120 when the system is in use. The venous return device 115 can be removably attached or fixedly attached to the shunt 120. The distal sheath 910 is adapted to be introduced through an incision or puncture into a venous return location, such as the jugular vein or femoral vein. The distal sheath 910 and flow line 915 can be permanently affixed, or can be attached using a conventional Luer fitting, as shown in FIG. 8A. Optionally, as shown in FIG. 8B, the sheath 910 can be joined to the flow line 915 by a Y-connector 1005. The Y-connector 1005 can include a hemostasis valve 1010. The venous return device also comprises a venous sheath dilator 1015 and an introducer guide wire 611 to facilitate introduction of the venous return device into the internal jugular vein or other vein. As with the arterial access dilator 645, the venous dilator 1015 includes a central guide wire lumen so the venous sheath and dilator combination can be placed over the guide wire 611. Optionally, the venous sheath 910 can include a flush line 1020 with a stopcock 1025 at its proximal or remote end.

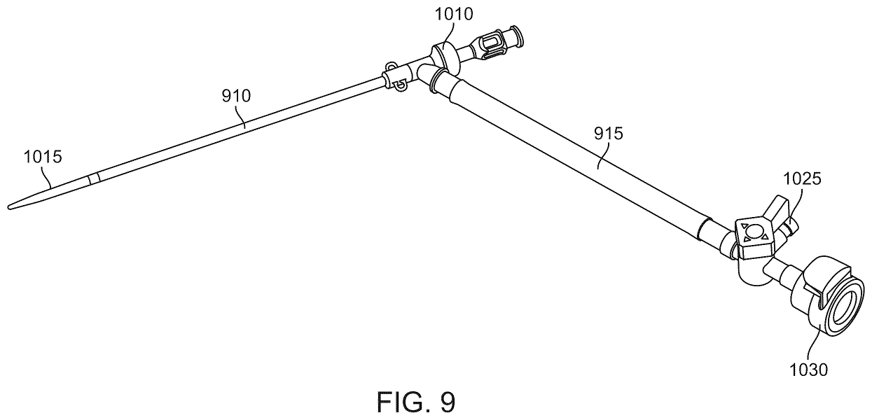

An alternate configuration is shown in FIGS. 8C and 9. FIG. 8C shows the components of the venous return device 115 including venous return sheath 910, sheath dilator 1015, and sheath guidewire 611. FIG. 9 shows the venous return device 115 as it would be assembled for insertion over the sheath guide wire 611 into a central vein. Once the sheath is inserted into the vein, the dilator and guidewire are removed. The venous sheath can include a hemostasis valve 1010 and flow line 915. A stopcock 1025 on the end of the flow line allows the venous sheath to be flushed via the flow line prior to use. Connection to the flow shunt 120 is made with a connector 1030 on the stopcock 1025.

### Retrograde Shunt

The shunt 120 can be formed of a single tube or multiple, connected tubes that provide fluid communication between the arterial access device 110 and the venous return catheter 115 to provide a pathway for retrograde blood flow therebetween. As shown in FIG. 1, the shunt 120 connects at one end (via connector 127a) to the flow line 615 of the arterial access device 110, and at an opposite end (via connector 127b) to the flow line 915 of the venous return catheter 115.

In an implementation, the shunt 120 can be formed of at least one tube that communicates with the flow control assembly 125. The shunt 120 can be any structure that provides a fluid pathway for blood flow. The shunt 120 can have a single lumen or it can have multiple lumens. The shunt 120 can be removably attached to the flow control assembly 125, arterial access device 110, and/or venous return device 115. Prior to use, the user can select a shunt 120 with a length that is most appropriate for use with the arterial access location and venous return location.

In an implementation, the shunt 120 can include one or more extension tubes that can be used to vary the length of the shunt 120. The extension tubes can be modularly attached to the shunt 120 to achieve a desired length. The modular aspect of the shunt 120 permits the user to lengthen the shunt 120 as needed depending on the site of venous return.

As mentioned, the length of the shunt can be adjusted. In an implementation, connectors between the shunt and the arterial and/or venous access devices are configured to minimize flow resistance. In an implementation, the arterial access sheath 110, the retrograde shunt 120, and the venous return sheath 115 are combined to create a low flow resistance arterio-venous AV shunt, as shown in FIG. 1. As described above, the connections and flow lines of all these devices are optimized to minimize or reduce the resistance to flow.

Actual flow through the AV shunt when in use will further depend on the cerebral blood pressures and flow resistances of the patient. In an implementation, the system is configured to detect blood pressure and control blood flow based on blood pressure such as by shutting down or reducing pressure of a pump connected to the system.

### Flow Control Assembly-Regulation and Monitoring of Retrograde Flow

The flow control assembly 125 interacts with the retrograde shunt 120 to regulate and/or monitor the retrograde flow rate from the common carotid artery to the venous return site, such as the femoral vein, internal jugular vein, or to the external receptacle 130. In this regard, the flow control assembly 125 enables the user to achieve higher maximum flow rates than existing systems and to also selectively adjust, set, or otherwise modulate the retrograde flow rate. Various mechanisms can be used to regulate the retrograde flow rate. The flow control assembly 125 enables the user to configure retrograde blood flow in a manner that is suited for various treatment regimens, as described below.

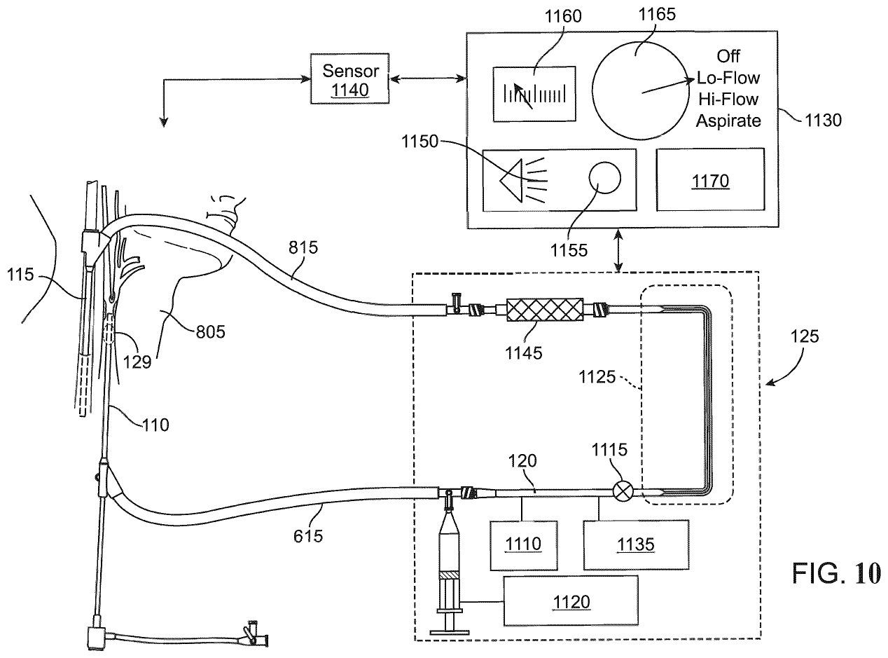

FIG. 10 shows an example of the system 100 with a schematic representation of the flow control assembly 125, which is positioned along the shunt 120 such that retrograde blood flow passes through or otherwise communicates with at least a portion of the flow control assembly 125. FIG. 10 shows a transcarotid access although the access location can vary and, as discussed, can be at the groin to achieve transfemoral access. The flow control assembly 125 can include various controllable mechanisms for regulating and/or monitoring retrograde flow. The mechanisms can include various means of controlling the retrograde flow, including one or more pumps 1110, valves 1115, syringes 1120 and/or a variable resistance component 1125. The flow control assembly 125 can be manually controlled by a user and/or automatically controlled via a controller 1130 to vary the flow through the shunt 120. For example, by varying the flow resistance, the rate of retrograde blood flow through the shunt 120 can be controlled. The controller 1130, which is described in more detail below, can be integrated into the flow control assembly 125 or it can be a separate component that communicates with the components of the flow control assembly 125.

In addition, the flow control assembly 125 can include one or more flow sensors 1135 and/or anatomical data sensors 1140 for sensing one or more aspects of the retrograde flow. A filter 1145 can be positioned along the shunt 120 for removing emboli before the blood is returned to the venous return site. When the filter 1145 is positioned upstream of the controller 1130, the filter 1145 can prevent emboli from entering the controller 1145 and potentially clogging the variable flow resistance component 1125. It should be appreciated that the various components of the flow control assembly 125 (including pump 1110, valves 1115, syringes 1120, variable resistance component 1125, sensors 1135/1140, and filter 1145) can be positioned at various locations along the shunt 120 and at various upstream or downstream locations relative to one another. The components of the flow control assembly 125 are not limited to the locations shown in FIG. 10. Moreover, the flow control assembly 125 does not necessarily include all of the components but can rather include various sub-combinations of the components. For example, a syringe could optionally be used within or outside of the flow control assembly 125 for purposes of regulating flow or it could be used outside of the assembly for purposes other than flow regulation, such as to introduce fluid such as radiopaque contrast into the artery in an antegrade direction via the shunt 120.

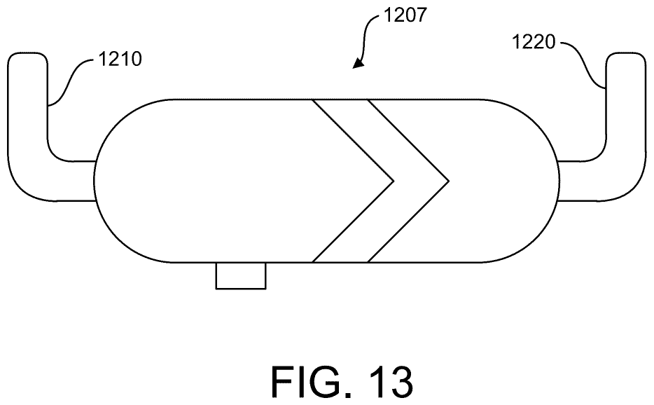

The flow control assembly 125 can vary in configuration. In an implementation, the flow control assembly includes a housing that is shaped to ergonomically interact with the patient's anatomy. For example, FIG. 13 shows a housing 1207 having a first port 1210 such as tubing with an internal lumen configured to communicate with an arterial access portion of the shunt and a second port 1220 such as tubing with an internal lumen configured to communicate with a venous access portion of a shunt. Either of the ports 1210 or 1220 can include or be coupled to a shape-set elbow portion configured to point or otherwise direct the shunt toward a predetermined location such as for a contralateral shunt. For example, one or both ports 1210 and 1220 can be L-shaped with a bend that can forms an angle of around 90 degrees in a non-limiting example.

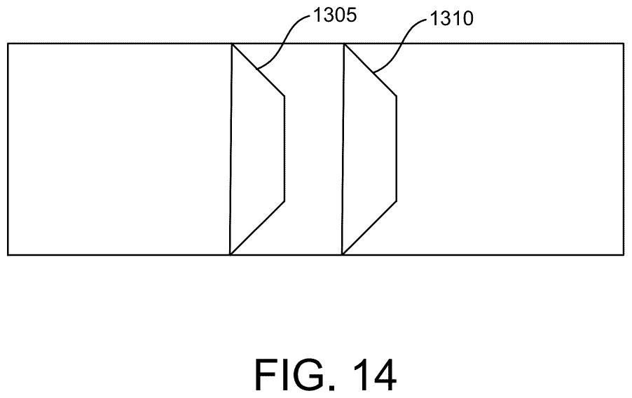

In an implementation, the filter 1145 includes two or more filters positioned in a layered arrangement along the blood flow pathway of the shunt. For example, as schematically shown in FIG. 14, a first filter 1305 and a second filter 1310 are positioned in series along a blood flow pathway of a blood vessel such that blood must flow through both filters as it flows through the shunt. The first filter 1305 can have at least a first pore size and the second filter has at least a second pore size different than the first pore size. The second pore size is larger than the first pore size such that the filters collectively filter out debris of different size. Any of the filters described herein can be part of a flow pathway that includes a bypass lumen wherein a user can cause blood to bypass the filter(s) such as in a situation where a filter is clogged.

Both the variable resistance component 1125 and the pump 1110 can be coupled to the shunt 120 to control the retrograde flow rate. The variable resistance component 1125 controls the flow resistance, while the pump 1110 provides for positive displacement of the blood through the shunt 120. Thus, the pump can be activated to drive the retrograde flow rather than relying on the perfusion stump pressures of the ECA and ICA and the venous back pressure to drive the retrograde flow. The pump 1110 can be a peristaltic tube pump or any type of pump including a positive displacement pump. The pump 1110 can be activated and deactivated (either manually or automatically via the controller 1130) to selectively achieve blood displacement through the shunt 120 and to control the flow rate through the shunt 120. Displacement of the blood through the shunt 120 can also be achieved in other manners including using the aspiration syringe 1120, or a suction source such as a vacutainer, vaculock syringe, or wall suction may be used. The pump 1110 can communicate with the controller 1130.

One or more flow control valves 1115 can be positioned along the pathway of the shunt. The valve(s) can be manually actuated or automatically actuated (via the controller 1130). The flow control valves 1115 can be, for example one-way valves to prevent flow in the antegrade direction in the shunt 120, check valves, or high pressure valves which would close off the shunt 120, for example during high-pressure contrast injections (which are intended to enter the arterial vasculature in an antegrade direction.

In an implementation of a shunt with both a filter 1145 and a one-way check valve 1115, the check valve is located downstream of the filter. In this manner, if there is debris traveling in the shunt, it is trapped in the filter before it reaches the check valve. Many check valve configurations include a sealing member that seals against a housing that contains a flow lumen.

The controller 1130 communicates with components of the system 100 including the flow control assembly 125 to enable manual and/or automatic regulation and/or monitoring of the retrograde flow through the components of the system 100 (including, for example, the shunt 120, the arterial access device 110, the venous return device 115 and the flow control assembly 125). For example, a user can actuate one or more actuators on the controller 1130 to manually control the components of the flow control assembly 125. Manual controls can include switches or dials or similar components located directly on the controller 1130 or components located remote from the controller 1130 such as a foot pedal or similar device. The controller 1130 can also automatically control the components of the system 100 without requiring input from the user. In an implementation, the user can program software in the controller 1130 to enable such automatic control. The controller 1130 can control actuation of the mechanical portions of the flow control assembly 125. The controller 1130 can include circuitry or programming that interprets signals generated by sensors 1135/1140 such that the controller 1130 can control actuation of the flow control assembly 125 in response to such signals generated by the sensors.

The representation of the controller 1130 in FIG. 10 is merely exemplary. It should be appreciated that the controller 1130 can vary in appearance and structure. The controller 1130 is shown in FIG. 10 as being integrated in a single housing. This permits the user to control the flow control assembly 125 from a single location. It should be appreciated that any of the components of the controller 1130 can be separated into separate housings. Further, FIG. 10 shows the controller 1130 and flow control assembly 125 as separate housings. It should be appreciated that the controller 1130 and flow control regulator 125 can be integrated into a single housing or can be divided into multiple housings or components.

### Flow State Indicator(s)

The controller 1130 can include one or more indicators that provides a visual and/or audio signal to the user regarding the state of the retrograde flow. An audio indication advantageously reminds the user of a flow state without requiring the user to visually check the flow controller 1130. The indicator(s) can include a speaker 1150 and/or a light 1155 or any other means for communicating the state of retrograde flow to the user. The controller 1130 can communicate with one or more sensors of the system to control activation of the indicator. Or activation of the indicator can be tied directly to the user actuating one of the flow control actuators 1165. The indicator need not be a speaker or a light. The indicator could simply be a button or switch that visually indicates the state of the retrograde flow. For example, the button being in a certain state (such as a pressed or down state) may be a visual indication that the retrograde flow is in a high state. Or a switch or dial pointing toward a particular labeled flow state may be a visual indication that the retrograde flow is in the labeled state.

The system can include an indicator to indicate to a user that blood flow has been stopped through the shunt or occluded in an artery.

### Flow Rate Actuators

The controller 1130 can include one or more actuators that the user can press, switch, manipulate, or otherwise actuate to regulate the retrograde flow rate and/or to monitor the flow rate. For example, the controller 1130 can include a flow control actuator 1165 (such as one or more buttons, knobs, dials, switches, etc.) that the user can actuate to cause the controller to selectively vary an aspect of the reverse flow. For example, in the illustrated implementation, the flow control actuator 1165 is a knob that can be turned to various discrete positions each of which corresponds to the controller 1130 causing the system 100 to achieve a particular retrograde flow state. The states include, for example, (a) OFF; (b) LO-FLOW; (c) HI-FLOW; and (d) ASPIRATE. It should be appreciated that the foregoing states are merely exemplary and that different states or combinations of states can be used. The controller 1130 achieves the various retrograde flow states by interacting with one or more components of the system, including the sensor(s), valve(s), variable resistance component, and/or pump(s). It should be appreciated that the controller 1130 can also include circuitry and software that regulates the retrograde flow rate and/or monitors the flow rate such that the user wouldn't need to actively actuate the controller 1130.

The OFF state corresponds to a state where there is no retrograde blood flow through the shunt 120. When the user sets the flow control actuator 1165 to OFF, the controller 1130 causes the retrograde flow to cease, such as by shutting off valves or closing a stop cock in the shunt 120. The LO-FLOW and HI-FLOW states correspond to a low retrograde flow rate and a high retrograde flow rate, respectively. When the user sets the flow control actuator 1165 to LO-FLOW or HI-FLOW, the controller 1130 interacts with components of the flow control regulator 125 including pump(s) 1110, valve(s) 1115 and/or variable resistance component 1125 to increase or decrease the flow rate accordingly. Finally, the ASPIRATE state corresponds to opening the circuit to a suction source, for example a vacutainer or suction unit, if active retrograde flow is desired.

The system can be used to vary the blood flow between various states including an active state, a passive state, an aspiration state, and an off state. The active state corresponds to the system using a means that actively drives retrograde blood flow. Such active means can include, for example, a pump, syringe, vacuum source, etc. The passive state corresponds to when retrograde blood flow is driven by the perfusion stump pressures of the ECA and ICA and possibly the venous pressure. The aspiration state corresponds to the system using a suction source, for example a vacutainer or suction unit, to drive retrograde blood flow. The off state corresponds to the system having zero or near zero retrograde blood flow such as the result of closing a stopcock or valve. The low and high flow rates can be either passive or active flow states. In an implementation, the particular value (such as in ml/min) of either the low flow rate and/or the high flow rate can be predetermined and/or pre-programmed into the controller such that the user does not actually set or input the value. Rather, the user simply selects “high flow” and/or “low flow” (such as by pressing an actuator such as a button on the controller 1130) and the controller 1130 interacts with one or more of the components of the flow control assembly 125 to cause the flow rate to achieve the predetermined high or low flow rate value. In another implementation, the user sets or inputs a value for low flow rate and/or high flow rate such as into the controller. In another implementation, the low flow rate and/or high flow rate is not actually set. Rather, external data (such as data from the anatomical data sensor 1140) is used as the basis for affects the flow rate.

The flow control actuator 1165 can be multiple actuators, for example one actuator, such as a button or switch, to switch state from LO-FLOW to HI-FLOW and another to close the flow loop to OFF, for example during a contrast injection where the contrast is directed antegrade into the carotid artery. In an implementation, the flow control actuator 1165 can include multiple actuators. For example, one actuator can be operated to switch flow rate from low to high, another actuator can be operated to temporarily stop flow, and a third actuator (such as a stopcock) can be operated for aspiration using a syringe. In another example, one actuator is operated to switch to LO-FLOW and another actuator is operated to switch to HI-FLOW. Or, the flow control actuator 1165 can include multiple actuators to switch states from LO-FLOW to HI-FLOW and additional actuators for fine-tuning flow rate within the high flow state and low flow state. Upon switching between LO-FLOW and HI-FLOW, these additional actuators can be used to fine-tune the flow rates within those states. Thus, it should be appreciated that within each state (i.e. high flow state and low flow states) a variety of flow rates can be dialed in and fine-tuned. A wide variety of actuators can be used to achieve control over the state of flow.

The controller 1130 or individual components of the controller 1130 can be located at various positions relative to the patient and/or relative to the other components of the system 100. For example, the flow control actuator 1165 can be located near the hemostasis valve where any interventional tools are introduced into the patient in order to facilitate access to the flow control actuator 1165 during introduction of the tools. The location may vary, for example, based on whether a transfemoral or a transcarotid approach is used. The controller 1130 can have a wireless connection to the remainder of the system 100 and/or a wired connection of adjustable length to permit remote control of the system 100. The controller 1130 can have a wireless connection with the flow control regulator 125 and/or a wired connection of adjustable length to permit remote control of the flow control regulator 125. The controller 1130 can also be integrated in the flow control regulator 125. Where the controller 1130 is mechanically connected to the components of the flow control assembly 125, a tether with mechanical actuation capabilities can connect the controller 1130 to one or more of the components. In an implementation, the controller 1130 can be positioned a sufficient distance from the system 100 to permit positioning the controller 1130 outside of a radiation field when fluoroscopy is in use.

The controller 1130 and any of its components can interact with other components of the system (such as the pump(s), sensor(s), shunt, etc) in various manners. For example, any of a variety of mechanical connections can be used to enable communication between the controller 1130 and the system components. Alternately, the controller 1130 can communicate electronically or magnetically with the system components. Electro-mechanical connections can also be used. The controller 1130 can be equipped with control software that enables the controller to implement control functions with the system components. The controller itself can be a mechanical, electrical or electro-mechanical device. The controller can be mechanically, pneumatically, or hydraulically actuated or electromechanically actuated (for example in the case of solenoid actuation of flow control state). The controller 1130 can include a computer, computer processor, and memory, as well as data storage capabilities.

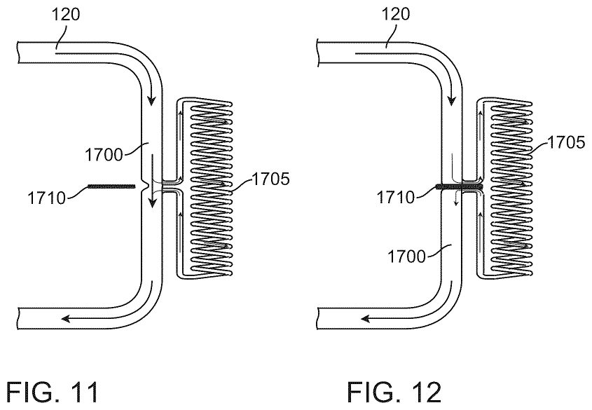

FIG. 11 shows an exemplary implementation of a variable flow control element 1125. In this implementation, the flow resistance through shunt 120 may be changed by providing two or more alternative flow paths to create a low and high resistance flow path. As shown in FIG. 11, the flow through shunt 120 passes through a main lumen 1700 as well as secondary lumen 1705 in parallel with the main lumen. The secondary lumen 1705 is longer and/or has a smaller diameter than the main lumen 1700. Thus, the secondary lumen 1705 has higher flow resistance than the main lumen 1700. By passing the blood through both these lumens, the flow resistance will be at a minimum. Blood can flow through both lumens 1700 and 1705 due to the pressure drop created in the main lumen 1700 across the inlet and outlet of the secondary lumen 1705. This has the benefit of preventing stagnant blood. As shown in FIG. 12, by blocking flow through the main lumen 1700 of shunt 120, the flow is diverted entirely to the secondary lumen 1705, thus increasing the flow resistance and reducing the blood flow rate. It will be appreciated that additional flow lumens could also be provided in parallel to allow for a three, four, or more discrete flow resistances. The shunt 120 may be equipped with a valve 1710 that controls flow to the main lumen 1700 and the secondary lumen 1705. The valve position may be controlled by an actuator such as a button or switch on the housing of flow controller 125.

In an implementation, the connectors which connect the elements of the reverse flow system are large bore, quick-connect style connectors. For example, a male large-bore hub 680 on the Y-adaptor 660 of arterial sheath 110 connects to a female counterpart on the arterial side of flow shunt 120. The connections can be standard female and male Luer connectors or other style of tubing connectors.

### Sensor(s)

As mentioned, the flow control assembly 125 can include or interact with one or more sensors, which communicate with the system 100 and/or communicate with the patient's anatomy. Each of the sensors can be adapted to respond to a physical stimulus (including, for example, heat, light, sound, pressure, magnetism, motion, etc.) and to transmit a resulting signal for measurement or display or for operating the controller 1130. In an implementation, the flow sensor 1135 interacts with the shunt 120 or the arterial access device to sense an aspect of the flow through the shunt 120, such as flow velocity or volumetric rate of blood flow. The flow sensor 1135 could be directly coupled to a display that directly displays the value of the volumetric flow rate or the flow velocity. Or the flow sensor 1135 could feed data to the controller 1130 for display of the volumetric flow rate or the flow velocity.

The type of flow sensor 1135 can vary. The flow sensor 1135 can be a mechanical device, such as a paddle wheel, flapper valve, rolling ball, or any mechanical component that responds to the flow through the shunt 120. Movement of the mechanical device in response to flow through the shunt 120 can serve as a visual indication of fluid flow and can also be calibrated to a scale as a visual indication of fluid flow rate. The mechanical device can be coupled to an electrical component. For example, a paddle wheel can be positioned in the shunt 120 such that fluid flow causes the paddle wheel to rotate, with greater rate of fluid flow causing a greater speed of rotation of the paddle wheel. The paddle wheel can be coupled magnetically to a Hall-effect sensor to detect the speed of rotation, which is indicative of the fluid flow rate through the shunt 120.

The system 100 is not limited to using a flow sensor 1135 that is positioned in the shunt 120 or a sensor that interacts with the venous return device 115 or the arterial access device 110. For example, an anatomical data sensor 1140 can communicate with or otherwise interact with the patient's anatomy such as the patient's neurological anatomy. In this manner, the anatomical data sensor 1140 can sense a measurable anatomical aspect that is directly or indirectly related to the rate of retrograde flow from the carotid artery. For example, the anatomical data sensor 1140 can measure blood flow conditions in the brain, for example the flow velocity in the middle cerebral artery, and communicate such conditions to a display and/or to the controller 1130 for adjustment of the retrograde flow rate based on predetermined criteria. In an implementation, the anatomical data sensor 1140 comprises a transcranial Doppler ultrasonography (TCD), which is an ultrasound test that uses reflected sound waves to evaluate blood as it flows through the brain. Use of TCD results in a TCD signal that can be communicated to the controller 1130 for controlling the retrograde flow rate to achieve or maintain a desired TCD profile. The anatomical data sensor 1140 can be based on any physiological measurement, including reverse flow rate, blood flow through the middle cerebral artery, TCD signals of embolic particles, or other neuromonitoring signals.

### Exemplary Methods of Use

Flow through the carotid artery bifurcation at different stages of the methods of the present disclosure will be described. Initially, the distal sheath 605 (or any implementation of the percutaneous sheaths described herein) of the arterial access device 110 is introduced into the common carotid artery CCA. As mentioned, entry into the common carotid artery CCA can be via a transfemoral approach such as via an access location at the groin and can be either a direct surgical cut-down or percutaneous access. Or entry can be directly in the CCA. Furthermore, alternate entry locations are possible including a transradial location. After the sheath 605 of the arterial access device 110 has been introduced into direct or indirect communication with the common carotid artery CCA, the blood flow will continue in antegrade direction AG with flow from the common carotid artery entering both the internal carotid artery ICA and the external carotid artery ECA.

The venous return device 115 is then inserted into a venous return site, such as the internal jugular vein IJV or femoral vein or brachial vein or subclavian vein, which are non-limiting examples. The shunt 120 is used to connect the flow lines 615 and 915 of the arterial access device 110 and the venous return device 115, respectively (as shown in FIG. 1). In this manner, the shunt 120 provides a passageway for retrograde flow from the atrial access device 110 to the venous return device 115. In another implementation, the shunt 120 connects to an external receptacle 130 rather than to the venous return device 115.

Once all components of the system are in place and connected, flow through the common carotid artery CCA is stopped, such as by using an expandable occlusion element of the percutaneous sheath in the common carotid artery CCA. Alternately, the occlusion element 129 is introduced on second occlusion device 112 separate from the distal sheath 605 of the arterial access device 110. The ECA may also be occluded with a separate occlusion element, either on the same device 110 or on a separate occlusion device.

At that point retrograde flow RG from the external carotid artery ECA and internal carotid artery ICA will begin and will flow through the sheath 605, the flow line 615, the shunt 120, and into the venous return device 115 via the flow line 915. The flow control assembly125 regulates the retrograde flow as described above. While the retrograde flow is maintained, a stent delivery catheter (or other intervention device) is introduced into the sheath 605. The stent delivery catheter is introduced into the sheath 605 through the hemostasis valve 615 and the proximal extension 610. The stent delivery catheter is advanced into the internal carotid artery ICA and a stent 2115 deployed at the bifurcation B.

Optionally, while flow from the common carotid artery continues and the internal carotid artery remains blocked, measures can be taken to further loosen emboli from the treated region. For example, mechanical elements may be used to clean or remove loose or loosely attached plaque or other potentially embolic debris within the stent, thrombolytic or other fluid delivery catheters may be used to clean the area, or other procedures may be performed. For example, treatment of in-stent restenosis using balloons, atherectomy, or more stents can be performed under retrograde flow. In another example, the occlusion balloon catheter may include flow or aspiration lumens or channels which open proximal to the balloon. Saline, thrombolytics, or other fluids may be infused and/or blood and debris aspirated to or from the treated area without the need for an additional device. While the emboli thus released will flow into the external carotid artery, the external carotid artery is generally less sensitive to emboli release than the internal carotid artery. By prophylactically removing potential emboli which remain, when flow to the internal carotid artery is reestablished, the risk of emboli release is even further reduced. The emboli can also be released under retrograde flow so that the emboli flows through the shunt 120 to the venous system, a filter in the shunt 120, or the receptacle 130.

After the lesion has been treated (e.g., using a stent) and the bifurcation has been cleared of emboli, the occlusion element 129 or alternately the tourniquet 2105 can be released, reestablishing antegrade flow. The sheath 605 can then be removed.

A self-closing element may be deployed about the penetration in the wall of the entry location, such as the common carotid artery or femoral artery, prior to withdrawing the sheath 605 at the end of the procedure. The self-closing element can be deployed at or near the beginning of the procedure, but optionally, the self-closing element can be deployed as the sheath is being withdrawn, often being released from a distal end of the sheath onto the wall of the common carotid artery.

### Flow Assistance System

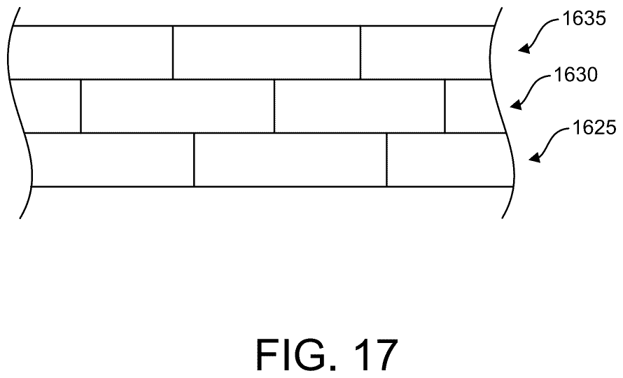

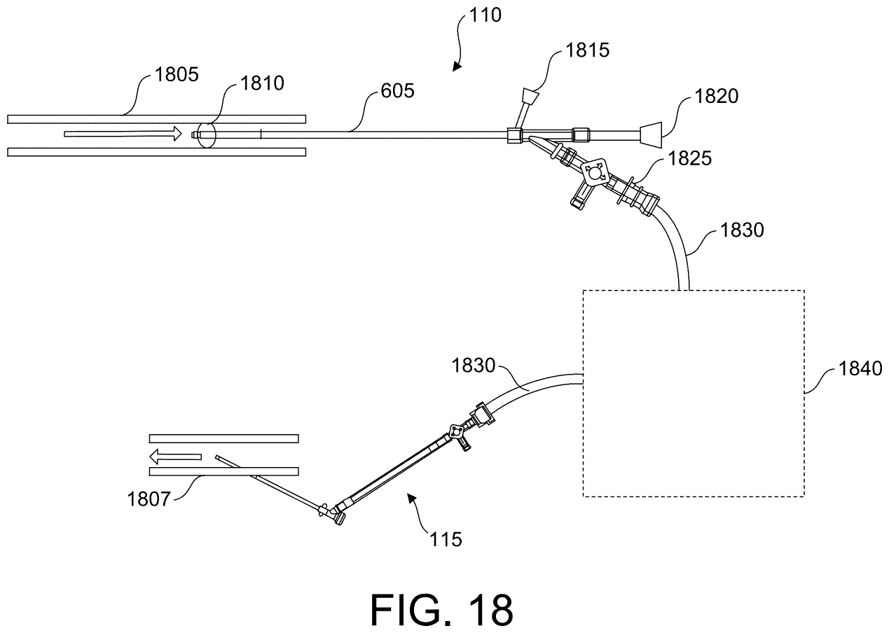

FIG. 18 shows a schematic representation of a flow assistance system configured to modify or increase the flow rate through the arterial access device and/or the shunt such as by increasing a pressure gradient across the shunt to increase fluid flow through the shunt. This can aid in embolic capture during tracking and placement of the catheter and for flow reversal once carotid vessel occlusion is achieved. The flow assistance system improves blood flow through a circuit that includes a high-pressure vessel (e.g., an artery), an external pump, a filter and flow circuit, and a return pathway to a low-pressure vessel (e.g., a vein) via a venous catheter.

FIG. 18 shows a schematic representation of the arterial access device 110 with a distal catheter or sheath 605 percutaneously positioned in the artery 1805, such as the common carotid artery, internal carotid artery, aorta or any peripheral artery such as via a transfemoral approach. Blood flow through a respective artery 1805 or vein 1807 is represented by an arrow. An occlusion balloon 1810 on the distal sheath 605 occludes the artery 1805. The arterial access device 110 can include various components such as an occlusion port 1815 (for inflating the occlusion balloon 1810), an interventional port 1820 (for inserting an interventional device and/or therapeutic agent), and a shunt port 1825 that connects to a reverse flow shunt formed of one or more items of tubing 1830. The shunt fluidly communicates with a venous return device 115 positioned in a vein 1807 such as for example the femoral vein or jugular vein. The dimensions of the arterial access device 110 can vary. In an example implementation, the distal sheath 605 has an internal lumen with an inner diameter of 0.070 inch to 0.0145 inch. In another example, the inner lumen is 0.084 inch to 0.0113 inch (6 Fr to 8 Fr. Inner diameter). In another example, the distal sheath 605 has a working length between 8 cm and 120 cm for peripheral access (transfemoral or transradial) or a working length of 8-20 cm for transcarotid access.

With reference still to FIG. 18, an external flow circuit 1840 is positioned along the shunt or incorporated directly in the shunt. The external flow circuit 1840 includes one or more elements configured to increase or otherwise adjust flow through the shunt, as described in more detail below. The external flow circuit 1840 can also include one or more elements configured to protect the patient from potential risk conditions. Such elements can include for example one or more valves, filters, flow rate controllers, etc. The external flow circuit 1840 can include a pump that modulates flow rate through the shunt by reducing a pump inlet pressure and increasing a pump outlet pressure. The pump does not excessively damage blood through hemolysis from excessive shear and/or heat. The pump may be powered in a variety of manners including an electric motor. In an example implementation, the electric motor is powered by at least one battery (e.g., 3-24 V DC) or an AC power source (e.g., 110/220 V outlet) in conjunction with a voltage controller to control and stabilize the power supply. The pump may be an occlusive or non-occlusive pump when operating.

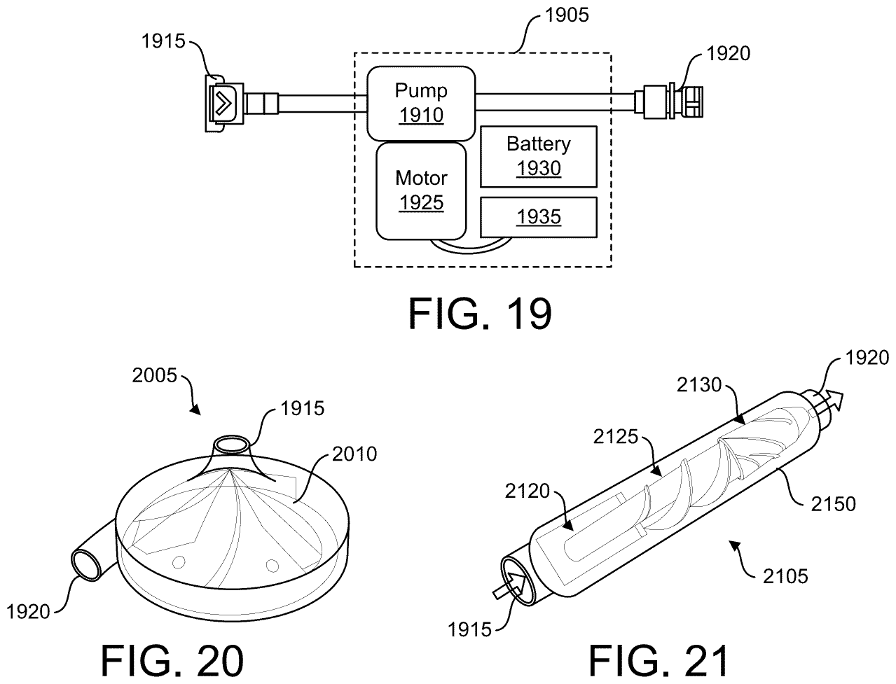

FIG. 19 shows a schematic representation of an example flow assist mechanism 1905 of the external flow circuit 1840. This implementation includes a pump 1910 having an inlet 1915 and an outlet 1920 each of which fluidly communicates with the shunt. The pump 1910 is coupled to a motor 1925 powered by a battery 1930 via a voltage circuit 1935. As shown in FIG. 20, the pump 1910 can be a centrifugal pump 2005 having an inlet 1915 and an outlet 1920 wherein an impeller 2010 is configured to selectively rotate to drive fluid therethrough.

As shown in FIG. 21, in another implementation the pump 1910 can be an axial pump 2105 having an inlet 1915 and an outlet 1920 wherein the axial body 2110 is configured to drive fluid therethrough. The axial body 2110 has a straightener section 2120, an impeller section 2125 and a diffuser section 2130. The straightener section is configured to achieve laminar blood flow while the diffuser section 2130 diffuses the blood flow. Any of the pump implementations have suction at the inlet and increased pressure at the outlet. The pressure delta across the pump can be increased by increasing the diameter and/or rotational speed of the impeller or axial body. In addition, blood flow can occur through the pump even while the pump is off.

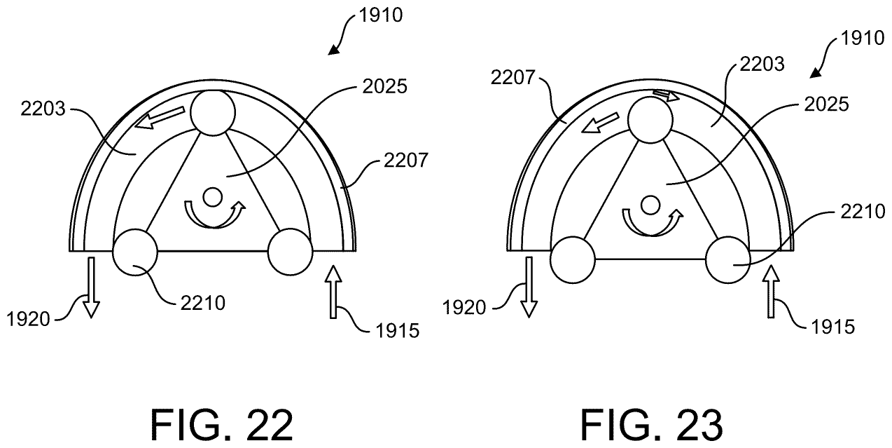

In another implementation, the pump 1910 is a roller pump. FIG. 22 schematically shows a pump 1910 comprising a roller pump with an inlet 1915 and an outlet 1920. A roller mechanism or rotor 2025 rotates about a central axis and has rollers 2210 that interact with a tubing 2203 having a blood flow lumen and wherein the tubing 2203 is part of the blood flow pathway of the shunt. The rotor 2025 rotates to successively move each roller 2210 into contact with the tubing. The rollers 2210 can be round, wheel structures that rotate about a central axis of the roller relative to the roller mechanism 2025. The rollers 2210 can have outer surfaces that mechanically interact with the tubing. The rollers are positioned to interact with the tubing 2203 such as by squeezing or otherwise deforming the tubing.

The tubing 2023 is flexible and can be coupled along its length to a pre-shaped structure 2207, such as a rigid structure, that causes the tubing 2023 to extend along a predetermined pathway with a predetermined shape such a, straight shape, a curved shape or a curvilinear shape in a region of the tubing that interacts with the rollers of the pump. The structure 2207 can extend along a portion of the tubing in a side-by-side relationship such that the flexible tubing 2203 conforms to the shape of the structure 2207 along the predetermined pathway. The roller mechanism 2025 rotates about an axis such that the rollers 2210 interact with the lumen of the tubing 2203 to pump blood through the lumen from the inlet 1915 toward the outlet 1920. Each roller 2210 is configured to rotate around a respective roller axis (which can be at the center point of each roller) so that each roller spins or rolls relative to the tubing when in contact with the tubing. Each roller can ride on a respective bushing to enable such rolling. Such action forms suction at the inlet 1915 and increased pressure at the outlet 1920. The rollers 2210 can be fully occlusive relative to the blood flow lumen. An electric motor can drive rotation of the roller mechanism 2025 and/or rollers. In another implementation shown in FIG. 23, the rollers 2210 are partially occlusive relative to the blood flow lumen of the tubing 2203. That is, the rollers 2210 do not completely occlude the lumen of the tubing when the rollers are positioned in contact with the tubing. For example, each roller can achieve 70% to 95% occlusion of the blood flow lumen in a non-limiting example to balance pumping efficiency with flow through.

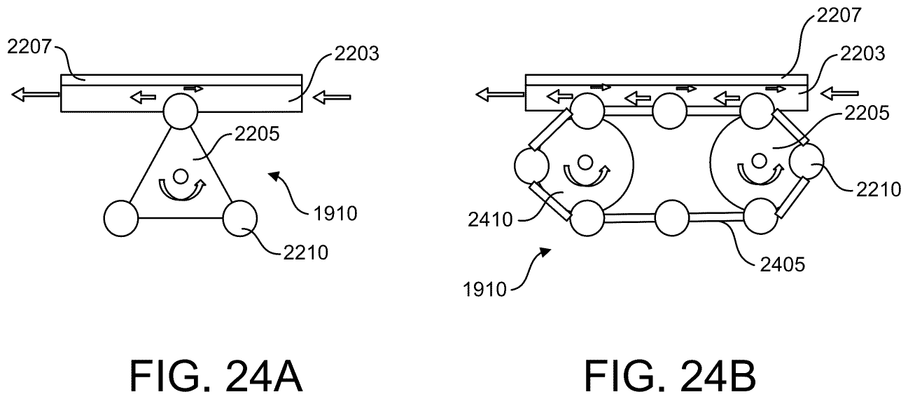

FIG. 24A shows another implementation of the pump 1910 as a roller pump wherein the tubing 2203 is straight in the region of the pump rather than curved as in the prior implementation. The structure 2207 can be straight and positioned adjacent and/or juxtaposed with the tubing 2203 to maintain the straight shape. FIG. 24B shows an implementation wherein the pump 1910 (as a roller pump) includes a series of rollers 2210 positioned on or otherwise coupled to an elongated roller pathway such as on a belt 2405. Each roller is coupled to the belt and the belt moves each roller into contact with the tubing. The rollers 2210 are also coupled to one or more rotors 2410 that rotate about respective axes at the center of each rotor 2410. Such rotation causes the belt 2405 and the attached rollers 2210 to move along the pathway of the belt to move the rollers 2210 along the elongated pathway of the belt 2405 relative to the blood flow lumen of the tubing 2203. This causes each roller 2210 to mechanically interact with the blood flow lumen such as to squeeze or otherwise deform the blood flow lumen and force blood to flow through the blood flow lumen.

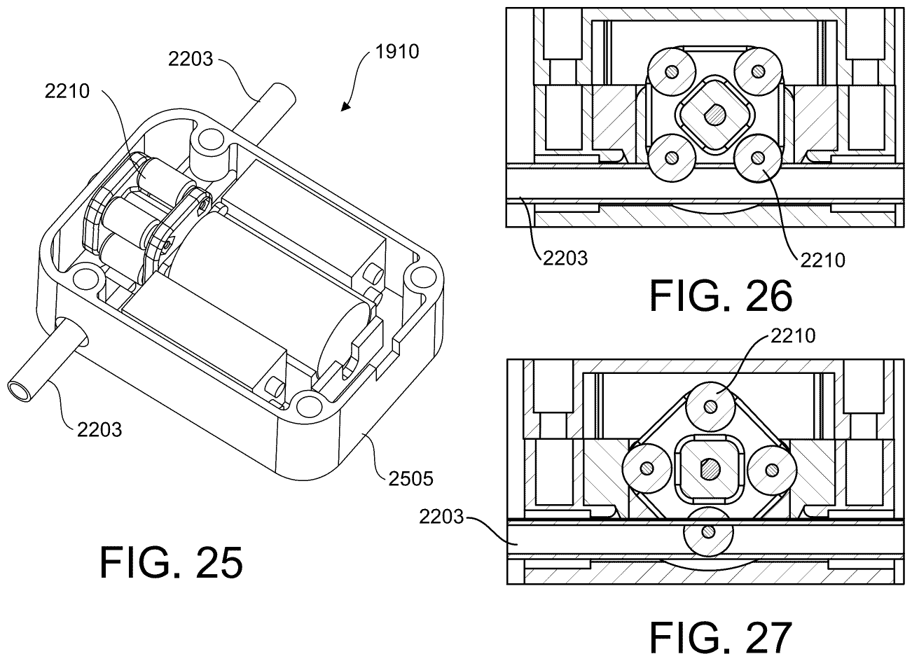

FIG. 25 shows another implementation of the pump 1910 wherein the pump is a roller pump or peristaltic pump that interacts with tubing 2203. The pump 1910 is positioned inside a housing 2505 where the tubing 2203 forms an inlet and an outlet of the pump. One or more rollers 2210 are positioned on a rotor assembly that rotates to variably position the rollers 2210 in communication with an internal lumen of the tubing 2203. A relative position between the rollers and the tubing can be adjusted to adjust a degree of occlusion that the roller achieves relative to an internal lumen of the tubing. For example, the rollers 2210 can be movably positioned and repositioned relative to the tubing to adjust the degree of occlusion that the rollers 2210 achieve relative to the internal lumen of the tubing 2203. FIG. 26 shows a view of the pump wherein the rollers 2210 are located in a position of minimum or less occlusion such that the rollers 2210 occlude only a portion of the internal lumen. FIG. 27 shows the rollers 2210 repositioned in a location relative to the tubing 2203 to achieve maximum or increased occlusion such that the rollers 2210 occlude an entirety of the flow pathway of the internal lumen. A size (e.g., diameter) of the rollers 2210 can be adjusted to modify a degree of occlusion. The tubing 2203 is shown as straight along the pump 1910 such as to achieve a flow pathway of less flow disturbance. The flow pathway can also be curved. In an implementation, the rollers 2210 can be locked at a desired degree of occlusion relative to the internal lumen of the tubing 2203. For example, the rollers 2210 can be locked at a minimal occlusion position when the pump is off to reduce flow resistance.

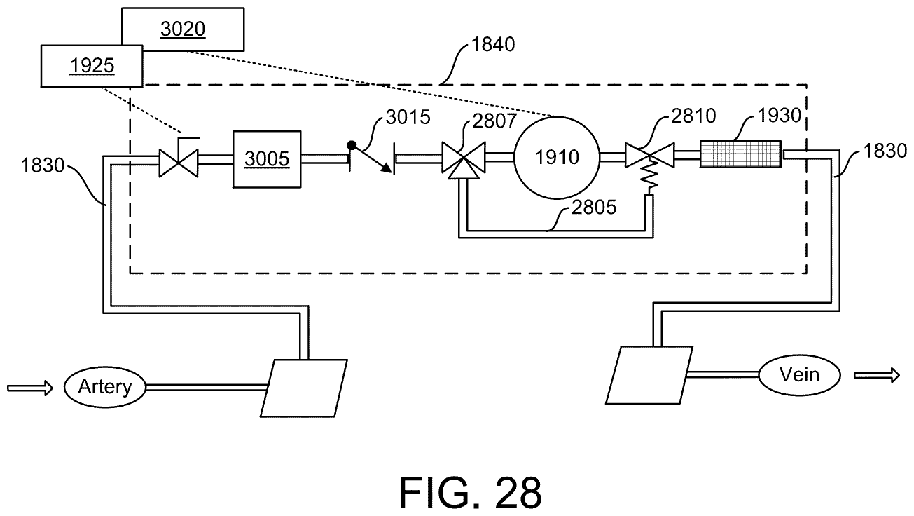

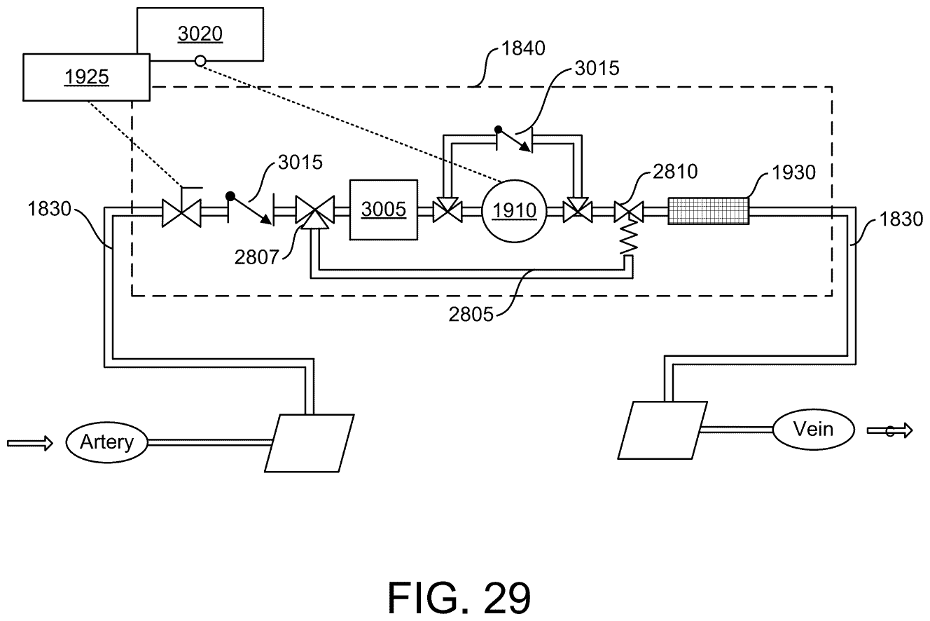

FIG. 28 and FIG. 29 show additional schematic representations of the flow assistance system. FIG. 28 shows an implementation that includes a pump 1910 that is a non-occlusive pump. FIG. 29 shows an implementation that includes a pump 1910 that is an occlusive pump.

With reference to FIGS. 28 and 29, the external flow circuit 1840 is positioned along the shunt (formed of tubing 1830.) The external flow circuit 1840 includes one or more elements configured to increase or otherwise adjust flow through the shunt, as described in more detail below. Each implementation includes a passive flow pathway 2805 wherein flow through the pathway 2805 is controlled by a valve 2807 wherein the passive flow pathway 2805 routes blood flow past the pump without assistance from the pump 1910. The passive flow pathway 2805 permits blood flow to recirculate in parallel around the pump 1910 such as in a situation where the pump is occluded (either intentionally or via an unintentional blockage.)

A pressure relief valve 2810 is positioned just past the pump 1910 in a flow direction and allows for blood flow to recirculate around the pump 1910 via the passive flow pathway 2805 such as where the pump 1910 is generating excessively high output pressures due to venous output blockage or a kink in a flow tubing 1830. The pressure relief valve opens at a predetermined pressure to redirect blood flow to the input of the pump 1910 in a situation where the outflow pressure increases beyond a set limit such as caused by occluded outflow. The pressure relief valve 2810 eliminates or reduces the situation where pressure is driven to unnecessarily high level.

The external flow circuit 1840 includes a vacuum flow resistor 3005, which is used in situations where the blood pressure near the arterial sheath is a strong vacuum and thus risks collapsing the artery. Implementations of the vacuum flow resistor 3005 are described in further detail below. The vacuum flow resistor 3005 collapses (to increase flow resistance) as a function of the pressure differential between the blood pressure in the shunt relative to atmosphere to reduce vacuum pressure experienced by the artery.

The external flow circuit 1840 includes at least one backflow preventer check valve 3015, which comprises a one-valve that prevents blood flow from inadvertently flowing from the flow circuit or vein back into the artery.

A high low switch 3020 is coupled to the pump 1910 and has a low flow setting a high flow setting. The external flow circuit 1840 further includes a stop switch 1925 (or on-off valve) that can be activated to temporarily stop blood flow through the system. A filter 1930 captures cloths and/or embolic debris. The serial order of components in the system may be modified or otherwise adjust performance.

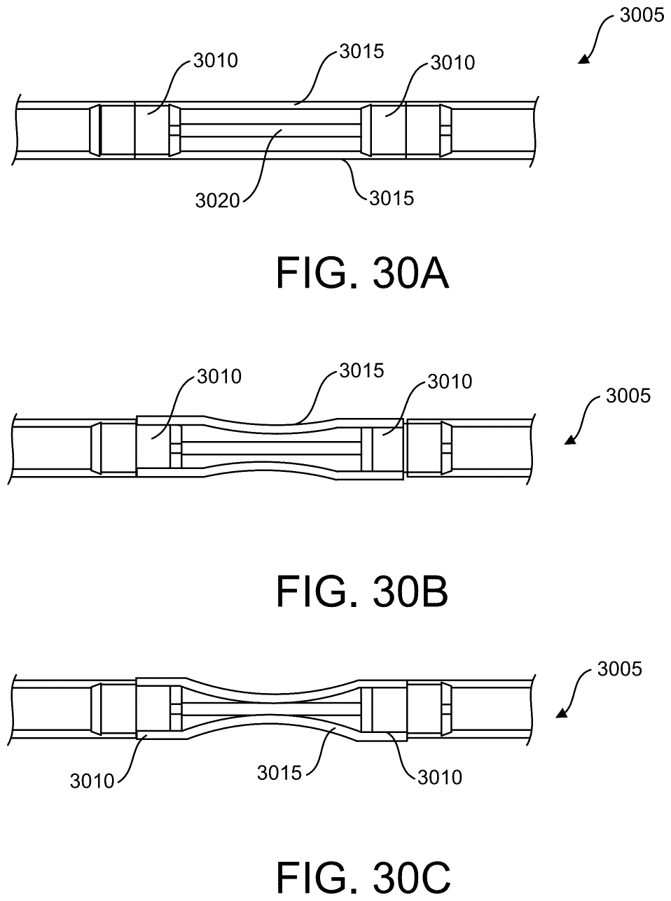

As discussed above, the system can include a vacuum flow resistor 3005. FIGS. 30A-30C show an implementation of a vacuum flow resistor 3005 that is positioned along the tubing 1830 of the shunt in the region of the external flow circuit. The vacuum flow resistor 3005 includes a pair of flow opposed connectors 3010 connected by a flexible outer tubing 3015 and an inner rigid tubing 3020. The flexible outer tubing 3015 forms a lumen through which blood flows. The rigid tubing 3020 is positioned within the lumen. As shown in FIGS. 30A-30C, the flexible outer tubing 3015 can change shape based upon a pressure differential wherein the shape change affects a level of flow resistance through the lumen, as described further below.

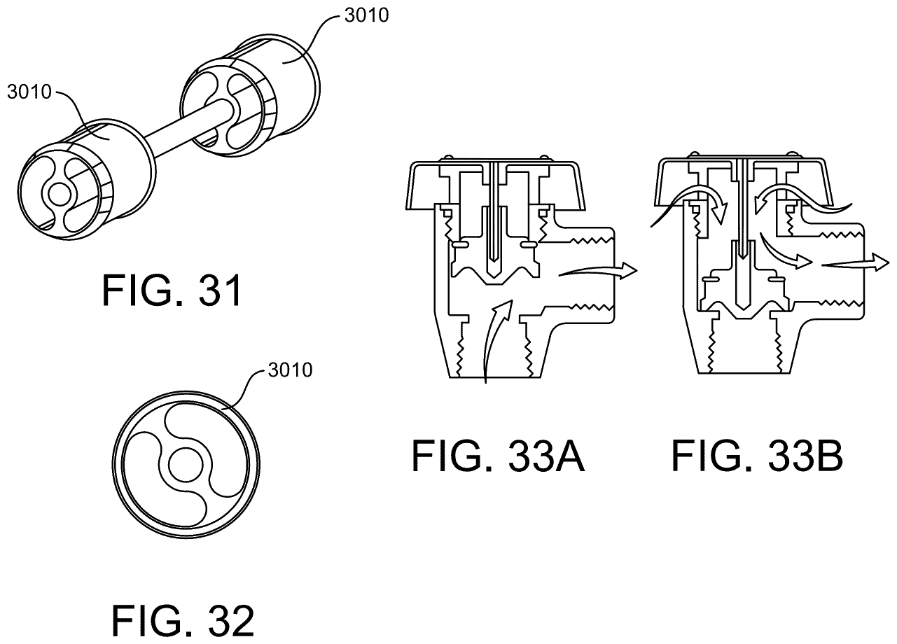

FIG. 31 shows a perspective view of the connectors 3010 and the rigid tubing 3020. FIG. 32 shows the connector 3010 as viewed along an axis of the rigid tubing 3020. The connector 3010 has one or more openings through which fluid (e.g., blood) can flow. The rigid tubing 3020 is an elongated body with a connector 3010 positioned at each end. The connectors 3010, rigid tubing 3020, and flexible outer tubing 3015 collectively form a flow pathway through which shunted blood flows.

With reference again to FIGS. 30A-30C, the vacuum flow resistor 3005 switches between an open state (where fluid flow can occur as in FIG. 30A) and a closed state (that blocks fluid flow as in FIG. 30C) based upon a pressure differential between the blood pressure Pi in the shunt and atmospheric pressure Patm. The vacuum flow resistor 3005 can also achieve a flow state between the open state and closed state as shown in FIG. 30B.

The flow resistance through the vacuum flow resistor 3005 increases as the pressure differential between the blood pressure Pi in the shunt and atmospheric pressure Patm increases. In FIG. 30A, the blood pressure Pi in the shunt is greater than or equal to atmospheric pressure Patm such that the flexible outer tubing 3015 forms an open lumen for blood to flow freely. As the pressure differential changes, meaning that the blood pressure Pi in the shunt becomes less than the atmospheric pressure Patm, as shown in FIG. 30B, the flexible outer tubing 3015 begins to collapse and reduces the size of the lumen therein to successively restrict blood flow therethrough. The blood flow rate thus slows down. As blood pressure Pi in the shunt becomes significantly less than atmospheric pressure Patm (as shown in FIG. 30C), the flexible outer tubing 3015 completely collapses to occlude blood flow therethrough. The diameter of the lumen formed by the flexible outer tubing 3015, as well as its length and its elastic properties dictate the extent of the flow resistance relative to the pressure data. A longer and more elastic tubing restricts blood flow more at a given pressure differential than a relatively shorter and stiffer tubing. Additionally, the flexible outer tubing 3015 may be sized such that its diameter is similar to that of the tube 3020 and thus restricts flow in all circumstances except for high input pressures. This could also be accomplished by an elastomeric/compliant ring that constricts tubing 3015.

FIGS. 33A and 33B show an implementation of a vacuum flow resistor 3005 that includes a vacuum breaker valve having an inlet and an outlet. FIG. 33A shows a scenario where the blood pressure in the shunt is greater than atmospheric pressure. In this scenario, the vacuum breaker valve is open to permit blood flow therethrough. FIG. 33B shows a scenario where the blood pressure in the shunt is less than atmospheric pressure. In this scenario, the vacuum breaker valve is closed to prohibit blood flow therethrough.

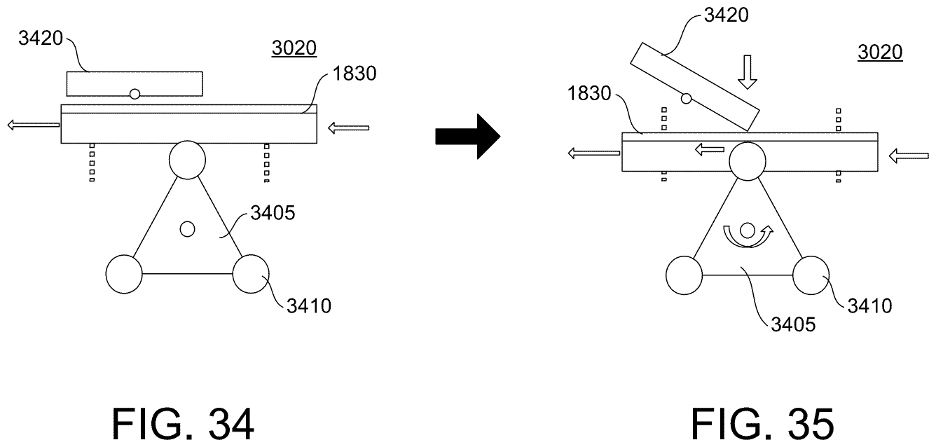

FIGS. 34 and 35 show an implementation of the high low switch 3020 configuration which is configured to switch flow between a low flow state (FIG. 34) and a high flow state (FIG. 35.) The high low switch 3020 is coupled to a pump that includes a rotor 3405 that rotates about an axis. The rotor 3405 includes one or more rollers 3410 wherein the rotor 3405 and the attached rollers 3410 can be positioned in a first position such that the rollers 3410 do not engage the tubing 1830 (as shown in FIG. 34) and a second position such that the rollers engage the tubing 1830 (as shown in FIG. 35.) A switch 3420 can be actuated to movably transition the tubing 1830 between the first position and the second position. In the first position, the tubing 1830 is positioned relative to the rotor 3405 so that the rollers 3410 do not (or minimally) contact the flow tubing 1830. Flow thus occurs relatively unimpeded. In the second position, the switch 3420 causes relative movement between the tubing 1830 and the rollers 3410 such that rollers 3410 can pump blood through the tubing 1830. The second position also results in powering of the pump such that the rollers pump blood through the tubing 1830 to increase flow rate.

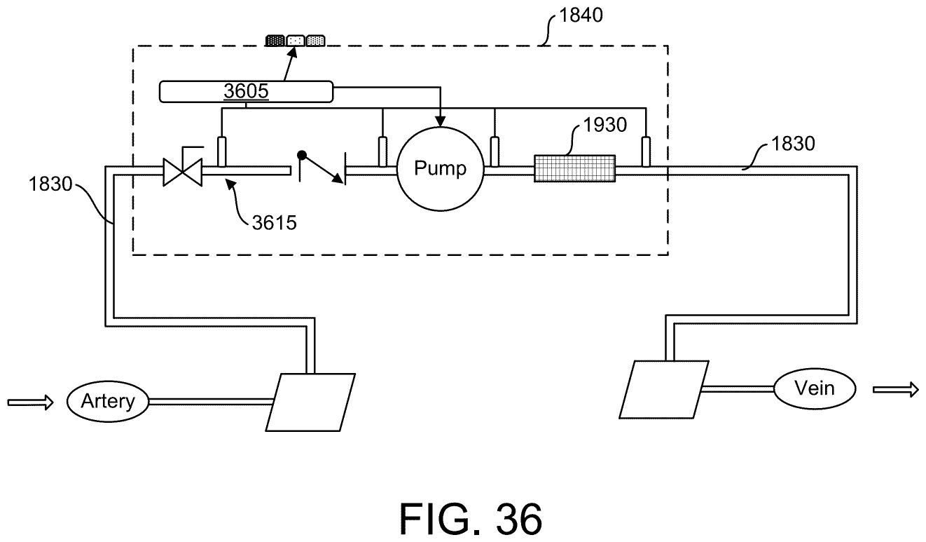

FIG. 36 shows a schematic representation of the flow assistance system wherein at least a portion of the external flow circuit is logic controlled. The external flow circuit 1840 includes a logic controller 3605 that is communicatively coupled to at least the pump 1910 and one or more pressure sensors 3615 positioned along the shunt. The pressure sensors can be located at various locations including for example immediately upstream of the pump, immediately downstream of the pump, and at each of the arterial catheter and venous catheter to monitor blood pressure at those locations. If pressure is sensed to drop below a threshold (e.g., such as indicative of a strong vacuum) or increase above a threshold (e.g., indicative of a flow pathway burst risk), the logic controller 3605 can modulate the pump 1910 (such as by modulating pump motor voltage) until the pressure or pressure delta achieves a desired pressure or falls within a desired pressure range. The system can include a visual or audio indicator to inform the user of the pressure state (e.g., low, medium high). The pump can also be logic controlled to a user defined setting (e.g., low, medium, high) or set to run for a predetermined amount of time.

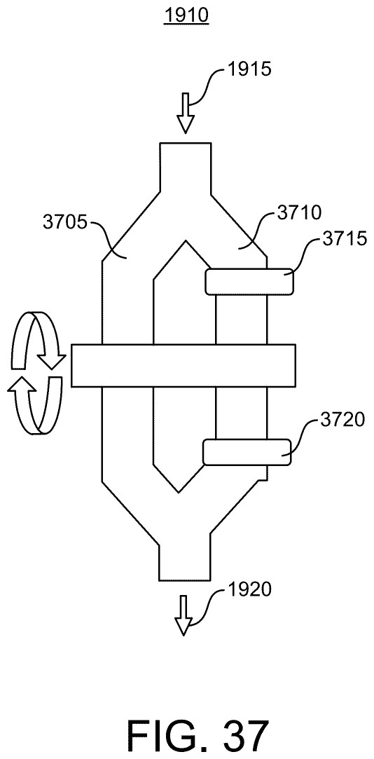

FIG. 37 shows a schematic representation of another pump 1910, which has an inlet 1915 and an outlet 1920 with a first flow pathway 3705 and a second flow pathway 3710 between the inlet 1915 and the outlet 1920. The first flow pathway 3705 serves as a primary flow pathway through the pump 1910 with no valves or restrictions through the first flow pathway 3705. The second flow pathway 3710 serves as a secondary flow pathway with one or more valves (e.g., check valves) or restrictions that govern flow through the pathway. The pump 1910 can include additional flow pathways beyond two flow pathways.

The second flow pathway 3710 includes an inlet valve 3715, an outlet valve 3720, and one or more secondary check valves. The inlet valve 3715 has a predetermined cracking pressure such as in the range of 1-3 psi (in a non-limiting example) to allow flow therethrough at high input pressure. At low input pressure, the pump 1910 utilizes only the first flow pathway 3705 as the inlet valve 3710 remains closed at such low pressure. The outlet valve 3720 has a relatively low cracking pressure configured to prevent backflow at low input pressure. At high input pressure, the pump 1910 utilizes both the first flow pathway 3705 and the second flow pathway 3710 as the inlet valve opens at such high pressure. The use of both the first flow pathway 3705 and the second flow pathway 3710 increases the pump efficiency. A desired efficiency, which can be predetermined and specifically tailored across a pressure input range can be controlled by adjusting the inlet valve cracking pressure and/or the size of the second flow pathway 3710 relative to the size of the first flow pathway 3705.

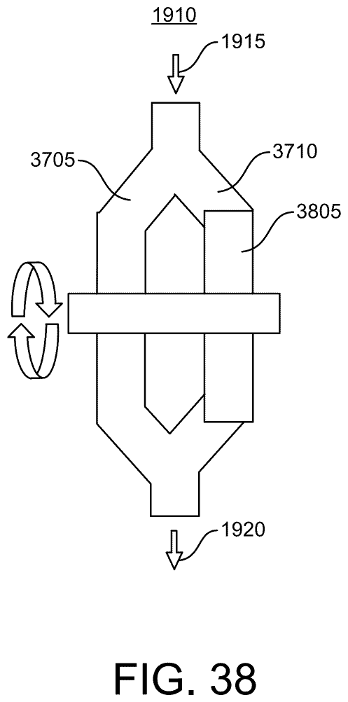

FIG. 38 shows another implementation of the pump 1910 that includes the first flow pathway 3705 and the second flow pathway 3710 between the inlet 1915 and the outlet 1920. The second flow pathway 3710 includes at least one high compliant portion 3805 made of a relatively high compliance material. The highly compliant material is such that the cross-sectional area (CSA) of the flow pathway increases as input pressure increases. The first flow pathway 3705 is made of a lower or moderate compliance material relative to the high compliant portion(s) 3805 of the second flow pathway 3710. At low input pressure, the second flow pathway 3710 has a smaller CSA due to the higher compliant material. At higher input pressure, the second flow pathway 3710 has a larger CSA to increase pump efficiency. The desired pump efficiency, which can be predetermined and specifically tailored across a pressure input range can be adjusted based on the relative compliance of the first flow pathway and the second flow pathway and the relative sizes of the first flow pathway and the second flow pathway.

Various techniques and modalities can be used to image the vascular anatomy during a procedure such as those described herein. Such techniques include, for example, computed tomography (CT) scan, ultrasound, and other surrogate imaging modalities. These methods generally translate forms of energy into visible light to create a visible image, which can be limit in various aspects, including resolution, depth of focus, field of view, and perspective. One difficulty with using visible light to view vascular anatomy, disease, treatment, trauma, and other features is that blood is not transparent. An injected bolus of saline can be used to clear blood from the visual field, but the resulting view is temporary and items such as turbulent flow and mixed blood can obscure the resulting view. Further, a given procedure may be incompatible with such a technique. Thus, current methods for intravascular imaging provide limited visual information that can make it difficult to fully understand vascular anatomy, disease, treatments, and trauma.

In order to visualize the inside of the vessel using optical devices (such as fiber optic endoscopes, scanning fiber endoscopes, optical coherence tomography, etc.), blood is flushed from the vessel. Injecting a bolus of saline through a flush line of the arterial access device during a flow stasis state can displace the blood from a region of interest and allow visualization using optical methods. Such a method of clamping or occluding the vessel as described herein, injecting saline (or other clear fluid such as contrast), and imaging can be done at any point with various results.

For example, evaluating the vascular anatomy or disease prior to introducing a angioplasty balloon or stent system helps to determine the position and extent of the lesion, Imaging can also be used after angioplasty and after stenting to evaluate the vessel or lesion following treatments. Flow reversal can utilized after imaging to ensure any debris that is generated during imaging is filtered using the techniques described herein.

Although implementations of various methods and devices are described herein in detail with reference to certain versions, it should be appreciated that other versions, implementations, methods of use, and combinations thereof are also possible. Therefore the spirit and scope of the appended claims should not be limited to the description of the implementations contained herein.

1. A system for use in accessing and treating a carotid artery, the system comprising:
an arterial access device adapted to be introduced into a common carotid artery via a percutaneous access location in a groin of a patient and receive blood flow from the common carotid artery via a distal region of the arterial access device, wherein the arterial access device includes a distal sheath which enters the common carotid artery;
a shunt fluidly connected to the arterial access device, wherein the shunt comprises tubing defining a blood flow pathway for blood to flow from the arterial access device to a return site; and
a flow control assembly coupled to the shunt and adapted to assist blood flow through the shunt, the flow control assembly including a pump having at least one roller that interacts with the tubing of the shunt to pump blood through the shunt toward the return site.

2. The system of claim 1, wherein the percutaneous access location is in a femoral vein.

3. The system of claim 1, wherein the at least one roller includes a plurality of rollers, each roller of the plurality of rollers coupled to a rotor that rotates to successively move each roller into contact with the tubing.

4. The system of claim 3, wherein each roller is coupled to a belt that forms an elongated pathway upon which each roller moves into contact with the tubing.

5. The system of claim 3, wherein the tubing of the shunt extends along a pathway having a predetermined shape, wherein the predetermined shape is straight or curved.

6. The system of claim 5, wherein a rigid structure extends along a portion of the tubing in a side-by-side relationship such that the tubing conforms to the shape of the structure.

7. The system of claim 1, wherein a relative position between the roller and the tubing can be adjusted to adjust a degree of occlusion that the roller achieves relative to an internal lumen of the tubing.

8. The system of claim 1, further comprising a dilator formed of an elongated body sized and shaped to be inserted into the arterial access device.

9. The system of claim 8, wherein the dilator has a first tapered region at a distal location of the dilator.

10. The system of claim 8, wherein the dilator has a second tapered region at a distal location of the dilator.

11. The system of claim 8, wherein the dilator has an optical marker on a proximal region of the dilator and wherein, when the optical marker aligns with a landmark of the arterial access device, the first tapered region fully extends from a distal tip of the arterial access device.

12. The system of claim 11, wherein alignment between the optical marker and the landmark of the arterial access device indicates to a user that a location where the dilator begins to taper is aligned with a distal most end of arterial access device.

13. The system of claim 1, wherein the arterial access device further comprises an expandable balloon on a distal region of the sheath, the expandable balloon adapted to occlude the common carotid artery.

14. The system of claim 13, wherein the sheath is formed of an outer jacket co-axially positioned over an inner layer, and wherein a distal portion of the balloon is connected to one of the outer jacket or the inner layer and a proximal portion of the balloon is connected to the other of the outer jacket or the inner layer.

15. The system of claim 13, wherein the sheath is formed of an outer jacket co-axially positioned over an inner layer, and wherein a distal portion of the balloon is connected to the inner layer and a proximal portion of the balloon is connected to the outer jacket.

16. A method of treating a carotid artery, comprising:
percutaneously accessing a femoral artery by inserting an arterial access device into the femoral artery through a puncture in the femoral artery, the arterial access device comprises a distal sheath;
introducing the distal sheath into a common carotid artery via an aortic arch;
expanding a balloon on the distal sheath to occlude at least a portion of the common carotid artery to cause blood to flow in a retrograde direction from an internal carotid artery into the distal sheath;
shunting blood from the sheath to a return site via a shunt fluidly coupled to the distal sheath; and
assisting blood flow through the shunt using a pump having at least one roller that interacts with a tubing of the shunt to pump blood through the shunt toward the return site.

17. The method of claim 16, wherein the at least one roller includes a plurality of rollers, each roller of the plurality of rollers coupled to a rotor that rotates to successively move each roller into contact with the tubing.

18. The method of claim 17, wherein each roller is coupled to a belt that forms an elongated pathway upon which each roller moves into contact with the tubing.

19. The method of claim 17, wherein the tubing of the shunt extends along a pathway having a predetermined shape, wherein the predetermined shape is straight or curved.

20. The method of claim 17, further comprising adjusting a relative position between the roller and the tubing to adjust a degree of occlusion that the roller achieves relative to an internal lumen of the tubing.
